(19) United States
(12) Patent Application Publication
(10) Pub. No.: US 2025/0011809 A1
(43) Pub. Date: Jan. 9, 2025

(54) VIRAL VECTOR PRODUCTION

(71) Applicants: Cell Therapy Catapult Limited, London (GB); pHion Therapeutics Ltd., Belfast (GB)

(72) Inventors: Jonathan APPLEBY, London (GB); Gregory BERGER, London (GB); Natacha AGABALYAN, London (GB); Matthew SMART, London (GB); Tristan THWAITES, London (GB); John MCBRIDE, Belfast (GB); Helen MCCARTHY, Belfast (GB)

(21) Appl. No.: 18/564,427

(22) PCT Filed: May 27, 2021
(86) PCT No.: PCT/GB2021/051310

§ 371 Date: Nov. 27, 2023

(51) Int. Cl. C12N 15/86; C07K 14/00

(52) U.S. Cl. CPC C12N 15/86; C07K 14/00; C12N 2750/14143; C12N 2750/14152

(57) ABSTRACT

This invention relates to the production of viral vectors. More specifically, the invention relates to amphipathic cell penetrating peptides for use in the production of viral vectors, and methods of using such peptides, as well as populations of viral vectors produced using such peptides.

### FIELD OF THE INVENTION

This invention relates to the production of viral vectors. More specifically, the invention relates to amphipathic cell penetrating peptides for use in the production of viral vectors, and methods of using such peptides, as well as populations of viral vectors produced using such peptides.

### BACKGROUND TO THE INVENTION

Viral vectors are central to the rapidly expanding field of clinical gene therapy, for vaccines and for oncolytic viral therapy. Viral vectors are also commonly used in research to deliver nucleic acids into cells. The progress from pre-clinical research, through clinical trials and towards market authorisation for advanced therapeutic medicinal products (ATMPs) of all types presents manufacturing challenges, including in relation to ensuring good manufacturing practice (GMP) to allow the production of well-characterised and consistent vector products that meet clinical standards for purity, potency and safety. In addition, viral vector manufacturing capacity is also an issue to ensure that sufficient quantities can be produced to allow therapeutically-effective doses to be available for use at scale in the clinic.

Although many advances have been made in viral vector design, barriers such as a pre-existing immune response can necessitate the administration of high vector titres and, in some cases, a combined administration of an immune-suppressant to achieve clinical efficacy. This presents a significant challenge in viral vector production.

Viral vectors are most commonly produced by a transient co-transfection of plasmids into a producer cell line. Significant progress has recently been made in large scale production and robust purification of viral vectors to support clinical development. However, production of high titre viral vectors is still a significant challenge, requiring patients to receive repeated administrations of a vector to achieve the desired dosage. For example, the dosing for Glybera, the first adeno-associated virus (AAV) vector approved by the European Medicines Agency was set at 1×10¹² vg/kg via 27 to 60 multiple injection sites. Other types of viral vector, such as lentiviral vectors, are also of clinical interest. For example, at the time of writing there are 143 clinical trials on the clinicaltrials.gov website listed as currently recruiting, enrolling or active that are investigating lentiviral gene therapy vectors for diseases ranging from chronic granulomatous disease, X-linked severe combined immunodeficiency and sickle cell disease. Issues with the production of viral vaccines have also been highlighted in recent times in the context of the AstraZeneca COVID-19 vaccine, which uses the modified chimpanzee adenovirus ChAdOx1 as a viral vector.

Furthermore, as a result of current purification methods, viral vector products typically contain high levels of protein aggregates or incompletely packaged empty capsids that lack vector DNA. The empty capsids in final products can often be as high as 60-fold over the level of complete particles. These impurities can trigger unwanted immune responses in patients. For example, recent studies have shown that cellular immune responses in mice and in human are directed to epitopes in the AAV2 capsids, and the presence of empty capsids inhibits hepatocyte transduction in vivo following high dose vector administration. Adverse immune responses, including increased IFNα and IFNβ production have also been reported for lentiviral gene therapy vectors. The potential adverse effects of the unwanted immunogenicity of empty viral particles compromise product safety and efficacy. It would therefore be desirable to minimise the proportion of empty viral capsids within a preparation of a viral vector, to minimise the dose needed to achieve a therapeutic effect.

Removal of empty capsids that have no therapeutic function by known methods can be difficult due to the innate similarity of their particle size, affinity and protein composition to the complete particles containing vector DNA, and is an active area of research for many companies working in the viral vector space. For example, ion exchange chromatography has also been reported for the separation of empty capsids in AAV2, 4, 5 and 8. However, from 20% up to 30 fold empty capsids remained in the final products.

Lentiviral manufacture has its own challenges, and whilst the specific question of full:empty ratios does not apply in this context, there will be a proportion of the manufactured lentiviral vector particles that do possess the desired characteristics, in particular may be defective and/or non-infective. Improvements to the methods for the production of lentiviral vectors such that a population with more favourable functional characteristics (in particular a higher proportion of infectious particles) is an active area of research.

These problems, whilst exemplified above in the context of AAV and lentiviral vectors, are not limited to the production of AAV or lentiviral vectors, but apply more generally to the production of viral vectors.

Therefore, there is an ongoing need for the development of production methods which can be effectively scaled to allow for the production of high titres of viral vectors to facilitate clinical use, and/or to reduce the cost of vector production. There is also a need to develop new methods of viral vector production which decrease or eliminate the presence of empty capsids in the final product. This would improve the safety and efficacy of viral vector products. Reduction of empty particles would also help overcome the hurdle in high titre production. It is an object of the present invention to overcome one or more of these issues.

### SUMMARY OF THE INVENTION

The present inventors have shown for the first time that an amphipathic cell penetrating peptide can be used in the production of viral vectors. In particular, the present inventors have shown for the first time that the amphipathic cell penetrating peptide of SEQ ID NO: 1 is capable of transfecting HEK 293T cells with multiple plasmids to produce AAV and lentiviral vectors. The inventors have also surprisingly shown that use of the RALA peptide provides multiple benefits over conventional transfection protocols, such as the use of polyethylenimine (PEI), which is the industry standard. In particular, the present inventors have demonstrated that RALA peptides are less cytotoxic to the producer cells than PEI, and that transfection efficiency using RALA peptides is increased compared with protocols using PEI. Significantly, the present inventors have shown that the use of RALA for transfection not only results in increased transfection efficiency, but also that the ratio of full:empty AAV particles is significantly increased using RALA peptides compared with protocols using PEI.

The use of amphipathic cell penetrating peptides as the transfection reagent, rather than conventional reagents such as PEI also has other significant advantages. In particular, the peptides themselves, and nanoparticles formed by complexing the peptides with the nucleic acids for transfection, can be readily lyophilised. In a lyophilised state, the peptides and nanoparticles are stable at room temperature, making them suitable for long-term storage, which is commercially desirable. In particular, this facilitates the use of such vectors from the supply chain up, because the lyophilised peptides and nanoparticles can be stored at room temperature, such that refrigerators or other cold storage is not required, simplifying the logistics around manufacture, distribution and storage.

In addition, industry standard protocols are typically based on adherent cell culture, using systems such as HYPERFlask® or HYPERStack®, or fixed-bed systems such as Icellis® bioreactors, as these facilitate superior producer cell growth. However, such adherent systems are often difficult to scale, and it is difficult to control conditions within the system once a culture has been initiated. Furthermore, plasmid DNA is susceptible to shear stresses, linked to its supercoiled topology creating an issue with torsion of the molecule. This susceptibility is relative to plasmid size, wherein the larger the plasmid, the more susceptible it is to shear stress degradation. Stirring during suspension culture, for example using stirred tank reactors (STRs) generates a degree of shear stress, which has the potential to impact on plasmid DNA.

Complexing the plasmids required for transfection through encapsulation using amphipathic cell penetrating peptides reduces their hydrodynamic size to below 100 nm. Without being bound by theory, it is believed that this reduces the relative force experienced by the plasmids, increasing their stability. In addition to the advantages this provides in terms of storage and shelf-life (see above), the inventors have also demonstrated that the use of RALA peptides advantageously allows for the successful production of viral vectors using suspension culture.

Using AAV8 for exemplification, the present inventors have shown that significant viral vector production is released into the culture medium. Combined with the inventors' surprising demonstration that RALA peptides allow for the efficient production of viral vectors in suspension culture, the inventors' methods have the potential to allow for the production of viral vectors using a continuous production system, without requiring harvest and lysis of the producer cells to extract the vector product.

A method which produces viral vectors at high titres and with increased full:empty vector ratios compared with conventional protocols offers significant advantages for the commercial production of viral vectors. Scalable suspension cultures and/or continuous cultures offering these advantages would be particularly desirable.

Accordingly, the present invention provides the use of an amphipathic cell penetrating peptide in the production of a viral vector, wherein the peptide is: a) less than approximately 50 amino acid residues in length; and b) has at least 6 arginine residues (R), at least 12 alanine residues (A), at least 6 leucine residues (L), optionally at least one cysteine residue (C), and at least two but no greater than three glutamic acids (E); wherein: (i) the arginine (R) residues are evenly distributed along the length of the peptide; the ratio of arginine (R) to negatively charged glutamic acid (E) residues is from at least 6:2 to 9:2 or 6:2 to 8:2; the ratio of hydrophilic amino acid residues to hydrophobic amino acid residues at pH 7 is at least 30:67 to 40:60 or 30:70 to 40:60; and said peptide comprises or consists of one of the following amino acid sequences: WEARLARALARALARHLARALARALRACEA (SEQ ID No. 1), WEARLARALARALARLARALARALRACEA (SEQ ID No. 2), WEARLARALARALARLARALARALRACEA (SEQ ID No. 3), REARLARALARALARLARALARALRACEA (SEQ ID No. 5), REARLARALARALARLARALARALRAREA (SEQ ID No. 6), REARLARALARALARELARALARALRAREA (SEQ ID No. 7), or a fragment thereof; (ii) said peptide comprises or consists of WEARLARALARALARELARALARALRACEA (SEQ ID No. 4), or (iii) said peptide comprises or consists of a peptide with at least 80% sequence identity to SEQ ID NO: 1, or a fragment thereof. The peptide may comprise or consist of SEQ ID NO: 1, 2, 5, or a fragment thereof. The peptide may be used to transfect a population of producer cells with one or more nucleic acid, optionally one or more plasmid.

The invention further provides a method of producing a viral vector, said method comprising the steps of: a) transfecting a population of producer cells with one or more nucleic acid, optionally one or more plasmid, using an amphipathic cell penetrating peptide as defined in herein; and b) harvesting the viral vectors produced by the transfected producer cells.

The peptide may be complexed with the one or more nucleic acid to form a nanoparticle prior to transfection, wherein optionally the peptide is complexed with two, three, four or five nucleic acids prior to transfection.

According to a use or method of the invention: a) each one or more nucleic acid may be complexed separately with the peptide to form nanoparticles, and optionally the nanoparticles for each one or more nucleic acid, are pooled prior to transfection; or b) the one or more nucleic acids may be pooled prior to complexing with the peptide to form nanoparticles.

The one or more nucleic acids may be plasmids.

According to a use or method of the invention: a) the transfection efficiency may be at least about 40%, preferably at least about 50%, more preferably at least about 60%, even more preferably at least about 70%; and/or b) the transfection may be at least two times more efficient, preferably at least three times more efficient, compared with a corresponding transfection using polyethylenimine (PEI).

According to a use or method of the invention: a) the peptide may result in cytotoxicity of less than 20%, preferably less than 10% to the producer cells; and/or b) the peptide may be at least 10 times, preferably at least 15 time, more preferably at least 20 times less toxic to the producer cells than PEI.

According to a use or method of the invention, the number of viral capsids produced may be at least 1.5-fold greater than the number of viral capsids produced compared with a corresponding transfection using PEI, preferably at least 2-fold greater, more preferably at least 3-fold greater.

According to a use or method of the invention: a) the ratio of empty:full viral particles may be about less than about 20:1, optionally about 15:1, about 10:1, about 8:1, about 6:1, about 2:1 or about 1:1; and/or b) the transfection may result in the production of at least about 30%, preferably at least about 40%, more preferably at least about 50%, even more preferably at least about 60%, more full viral particles compared with a corresponding transfection using PEI.

Any one or more of the advantages described herein may be obtained alone or in combination in the methods and uses of the invention.

The viral vector may be an adeno-associated virus (AAV) vector, a lentiviral vector, a retroviral vector, an adenoviral vector, a herpes-simplex viral vector, a poxvirus vector or a baculovirus vector. The viral vector may be an AAV vector, such as an AAV2, AAV8, AAV1, AAV3, AAV4, AAV5, AAV6, AAV7, AAV8, AAV9, AAV10 or AAV11 serotype, preferably an AAV2 or AAV8 serotype.

The producer cells may be: (a) mammalian cells, preferably human cells, optionally HEK293, HEK293-F or HEK293T cells; or (b) insect cells, optionally Sf9, Sf21 or S2 cells.

The one or more plasmid may comprise (i) a plasmid comprising the RepCap genes; (ii) a plasmid comprising a transgene of interest; and (iii) a helper plasmid.

Transfection of the producer cells may be carried out in serum-free or low-serum medium.

The population of producer cells may be cultured and transfected in a suspension culture system or an adherent culture system.

A use or method of the invention may be for the continuous production of viral vector, preferably an AAV vector or lentiviral vector, more preferably an AAV1, AAV2 or AAV8 serotype.

A method or use of the invention may further comprise one or more of the following steps: a) expanding the population of producer cells prior to transfection; b) culturing the population of producer cells prior to transfection; c) changing the medium of the producer cells prior to transfection; d) changing the medium of the producer cells after transfection; and/or e) purification of the viral vectors.

A method or use of the invention may comprise harvesting the viral vector from the culture medium and/or the transfected producer cells.

A method or use of the invention may not comprise a step of polishing the viral vectors, preferably wherein said method or use does not include an anion exchange polishing step, and/or an ultrafiltration polishing step.

The invention further provides a population of viral vectors obtainable by a method or use of the invention. The population of viral vectors may have a ratio of empty:full viral particles of about 20:1 or less, optionally about 15:1, about 10:1, about 8:1, about 6:1, about 2:1 or about 1:1.

### BRIEF DESCRIPTION OF THE DRAWINGS

FIG. 1: Graph to show % viability of HEK293 cells following transfection with PEI or RALA with a plasmid comprising a GFP reporter gene.

FIG. 2: Flowchart of AAV2 experiment of Example 2.

FIG. 3: Representative cell fluoroscopy images of GFP⁺ expression following transfection of HEK293 cells with AAV-GFP using PEI or RALA peptides under different conditions.

FIG. 4: AAV2 vector genome titres as measured by qPCR for RALA and PEI conditions. Error bars show standard deviation.

FIG. 5: AAV2 vector capsid titres as measured by ELISA (left panel) and % of full capsids (right panel) for RALA and PEI conditions. Error bars show standard deviation.

FIG. 6: Flowchart of AAV8 experiment of Example 3.

FIG. 7: AAV8 vector genome titres as measured by qPCR (left panel) and AAV8 vector capsid titres as measured by ELISA, with the proportion of full AAV8 capsids also shown (right panel) using RALA and PEI-Pro.

FIG. 8: AAV8 vector capsid titres as measured by ELISA (top row) in harvested media (left panel) and cell lysate (right panel) and AAV8 vector genome titres as measured by qPCR (bottom row) in harvested media (left panel) and cell lysate (right panel), using RALA and PEI-Pro.

FIG. 9: Flowchart of AAV8 experiment of Example 4.

FIG. 10: Daily AAV8 vector capsid titres (left panel) and cumulative AAV8 vector capsid titres (right panel) as measured by ELISA using RALA and PEI-Pro.

FIG. 11: Daily AAV8 vector genome titres (left panel) and cumulative AAV8 vector genome titres (right panel) as measured by qPCR using RALA and PEI-Pro.

FIG. 12: Graph to show % viability of producer cells over time following transfection with PEI or RALA for the production of LV, AAV1 and AAV2. Error bars show standard deviation.

FIG. 13: Graph to show producer cell counts over time following transfection with PEI or RALA for the production of LV, AAV1 and AAV2. Error bars show standard deviation.

FIG. 14: Daily Empty:full ratios of AAV8 particles released into the media.

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary.

This disclosure is not limited by the exemplary methods and materials disclosed herein, and any methods and materials similar or equivalent to those described herein can be used in the practice or testing of embodiments of this disclosure. The terminology used herein is for the purpose of describing particular embodiments only, and is not intended to limit the scope of the present invention, which is defined solely by the claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g. to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, 3%, 2%, 1%, 0.5%, 0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

As used herein, the terms “vector” and “viral vector” are used interchangeably and encompass adeno-associated virus (AAV) vectors, lentiviral vectors, retroviral vectors, adenoviral vectors, herpes-simplex virus (HSV) vectors, poxvirus vectors and baculovirus vectors. All disclosure herein in relation to viral vectors of the invention applies equally and without reservation to specific types of viral vectors of the invention (e.g. AAV vectors and/or lentiviral vectors) unless expressly stated otherwise.

As used herein, the term “capsid” refers to a protein coat which surrounds the nucleic acid cargo of the viral vector. Thus, the term encompasses both regular (e.g. icosahedral) capsids and other protein coat structures, such as helical protein sheaths. A lentivirus particle may be assumed to contain either 2000 Gag molecules or 2 viral RNA molecules.

As used herein, the terms “titre” and “yield” are used interchangeably to mean the amount of viral vector produced by a method of the invention. Titre is the primary benchmark characterising manufacturing efficiency, with higher titres generally indicating that more viral vector is manufactured (e.g. using the same amount of reagents). Titre or yield may relate either to the number of physical viral particles (i.e. the total number of viral particles) or “active” virus particles, i.e. the number of particles capable of transducing a cell. For example, the number of “physical” virus particles may be quantified using the number of viral genomes (DNA/RNA) produced by a method of the invention, which may be measured using any appropriate means, such as qPCR. Alternatively, the number of “active” particles capable of transducing a cell may be calculated. The total number of (full+empty) virus particles may also be determined using any appropriate means, such as by measuring either how much Gag is present in the test solution or how many copies of viral capsids are in a test sample. Typically the total number of (full+empty) virus particles may be determined by measuring the number of viral capsids produced, which can be measured using any appropriate technique, such as by titration ELISA. The ratio of full:empty viral particles may then be calculated based on assumptions regarding, e.g. the number of viral capsids, and/or viral genomes within a viral particle. An AVV particle may be assumed to contain one viral genome per AAV capsid (wherein the AAV capsid comprise VP1, VP2 and VP3 at a 1:1:10 ratio). For other viral vectors where the specific question of full:empty ratios does not apply calculating titre may comprise quantifying the number of defective and/or non-infective virus particles, rather than the number of full/empty particles. Any appropriate method may be used for such quantification, standard examples of which are known in the art. For example, physical titre may be measured via ELISA or qPCR. Functional titre may be used to quantify the number of infectious virus particles, for example by FACS to count the number of target cells that are positive for vector encoded transgene expression following transduction with serial dilutions of a viral preparation.

Amino acids are referred to herein using the name of the amino acid, the three-letter abbreviation or the single letter abbreviation.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including mRNA, tRNA, miRNA, sncRNA, LncRNA, siRNA, shRNA, and antisense oligonucleotides. The terms “transgene” and “gene” are also used interchangeably and both terms encompass fragments or variants thereof encoding the target protein.

The transgenes of the present invention include nucleic acid sequences that have been removed from their naturally occurring environment, recombinant or cloned DNA isolates, and chemically synthesized analogues or analogues biologically synthesized by heterologous systems.

Minor variations in the amino acid sequences of the invention are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the amino acid sequence of the invention or a fragment thereof as defined anywhere herein. The term homology is used herein to mean identity. As such, the sequence of a variant or analogue sequence of an amino acid sequence of the invention may differ on the basis of substitution (typically conservative substitution) deletion or insertion. Proteins comprising such variations are referred to herein as variants.

Proteins of the invention may include variants in which amino acid residues from one species are substituted for the corresponding residue in another species, either at the conserved or non-conserved positions. Variants of protein molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6]quantitative activity-property relationships of proteins can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN:1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of proteins can be derived from empirical and theoretical models (for example, analysis of likely contact residues or calculated physicochemical property) of proteins sequence, functional and three-dimensional structures and these properties can be considered individually and in combination.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a protein of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, lie, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

A “fragment” of a polypeptide comprises at least 50%, at least 60%, at least 70%, at least 80%, at least 90%, at least 95%, at least 97% or more of the original polypeptide. A “fragment” of a polypeptide may comprise at least 15, at least 20, at least 25, at least 26, at least 27, at least 28, at least 29 or more amino acids.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In view of the degeneracy of the genetic code, considerable sequence variation is possible among the polynucleotides of the present invention. Degenerate codons encompassing all possible codons for a given amino acid are set forth below:

| Amino | | Degenerate |
|---|---|---|
| Acid | Codons | Codon |
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/Asp | | RAY |
| Glu/Gln | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85, 90, 91, 92, 93, 94, 95, 96, 97, 98, 99 or more % of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species. Thus, according to the invention, in addition to the gag-pol genes any nucleic acid sequence may be codon-optimised for expression in a host or target cell. In particular, the vector genome (or corresponding plasmid), the REV gene (or corresponding plasmid), the fusion protein (F) gene (or correspond plasmid) and/or the hemagglutinin-neuraminidase (HN) gene (or corresponding plasmid, or any combination thereof may be codon-optimised.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment may include at least one antigenic determinant and/or may encode at least one antigenic epitope of the corresponding polypeptide of interest. Typically, a fragment as defined herein retains the same function as the full-length polynucleotide.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation) as compared to a reference level.

The terms “increased”, “increase”, “enhance”, or “activate” are all used herein to mean an increase by a statically significant amount. The terms “increased”, “increase”, “enhance”, or “activate” can mean an increase of at least 25%, at least 50% as compared to a reference level, for example an increase of at least about 50%, or at least about 75%, or at least about 80%, or at least about 90%, or at least about 100%, or at least about 150%, or at least about 200%, or at least about 250% or more compared with a reference level, or at least about a 1.5-fold, or at least about a 2-fold, or at least about a 2.5-fold, or at least about a 3-fold, or at least about a 4-fold, or at least about a 5-fold or at least about a 10-fold increase, or any increase between 1.5-fold and 10-fold or greater as compared to a reference level. In the context of a yield or titre, an “increase” is an observable or statistically significant increase in such level.

Herein the terms “control” and “reference population” are used interchangeably.

A control method may be any standard method known in the art for producing viral vectors, typically any standard method known in the art for the production of the same type of viral vectors as produced by the method of the invention. By way of non-limiting example, for a method of producing AAV vectors according to the invention, a control method would be any standard or conventional method of AAV vector production. By way of a further non-limiting example, for a method of producing lentiviral vectors according to the invention, a control method would be any standard or conventional method of lentiviral vector production. A preferred control method according to the invention uses PEI as a transfection reagent. A control method may be essentially identical to the method of the invention, but differ only in the transfection reagent used and associated parameters. By way of non-limiting example, a control method may use the same one or more nucleic acids (e.g. plasmids) and/or the same producer cell type compared with a method of the invention. Preferably a control method uses PEI compared with an amphipathic cell penetrating reagent as used in a method of the invention.

Similarly, a control reagent may be any standard reagent known in the art for producing viral vectors, typically any standard reagent known in the art for the production of the same type of viral vectors as produced by the method of the invention. A preferred control reagent is PEI.

Viral vectors and viral vector populations produced by such control/standard methods and/or reagents may be used as control vectors and populations as described herein.

The term “pharmaceutically acceptable” as used herein means approved by a regulatory agency of the Federal or a state government, or listed in the U.S. Pharmacopeia, European Pharmacopeia or other generally recognized pharmacopeia The publications discussed herein are provided solely for their disclosure prior to the filing date of the present application. Nothing herein is to be construed as an admission that such publications constitute prior art to the claims appended hereto.

Disclosure related to the various methods of the invention are intended to be applied equally to other methods, therapeutic uses or methods, the data storage medium or device, the computer program product, and vice versa.

### Amphipathic Cell Penetrating Peptides

The RALA family of peptides are amphipathic peptides composed of repeating RALA units that are capable of overcoming biological barriers to gene delivery, both in vitro and in vivo. The term “RALA” has been used inconsistently in the literature, but typically refers to an amphipathic peptide or group of peptides composed of repeating RALA units generally of less than approximately 50 amino acid residues. Cohen-Avrahami et al. (J. Phys. Chem. B 2011, 115:10 189-1 097 and Colloids and Surfaces B: Biointerfaces 77 (201 0) 131-138) disclose an amphipathic 16-mer peptide referred to as “RALA”. Faranack et al. (Biomacromolecules 2013, 14, 2033-2040) uses the term “RALA” to describe a 30-mer RALA peptide, as does McCarthy et al. (Journal of Controlled Release 189 (2014) 141-149) but for a different 30-mer peptide. WO 2014/087023 and WO 2015/189205 defined the term “RALA” as a generic term for a group of peptides falling within the scope of the invention as described therein.

The invention provides an amphipathic cell penetrating peptide (also referred to interchangeably herein as a “peptide”) for use in the production of a viral vector. Amphipathic cell penetrating peptides are described in detail in EP2,928,909B1, which is herein incorporated by reference in its entirety (with particular reference to paragraphs [0033] to [0047] thereof). The amphipathic cell penetrating peptide typically comprises or consists of an amphipathic cell penetrating peptide less than approximately 50 amino acid residues comprising at least 6 arginine residues (R), at least 12 alanine residues (A), at least 6 leucine residues (L), optionally at least one cysteine residue (C) and at least two but no more than three glutamic acids (E). The arginine (R) residues are typically evenly distributed along the length of the peptide. The ratio of arginine (R) to negatively charged amino acid residues glutamic acid (E) is typically from at least 6:2 to 9:2 or 6:2 to 8:2. Typically the ratio of hydrophilic amino acid residues to hydrophobic amino acid residues at pH 7 is at least 30:70 to 40:60 or 30:67 to 40:60. According to the invention, these peptides are RALA peptides, and the peptides inconsistently and ambiguously referred to as RALA peptides in the prior art do not fall within this definition.

The presence of arginine (R) residues in the amphipathic cell penetrating peptide is essential. Ensuring an even distribution of arginine (R) residues along the length of the peptide facilitates delivery of the peptide across a cell membrane by condensing the negatively charged compound or nucleic acid through electrostatic interactions. The presence of arginine (R) enables nanoparticles less than 20 nm to form and ensures a positive zeta potential which enables internalisation into the cell. The presence of arginine (R) residues also enhances nuclear localisation.

The ratio of the positively charged amino acid residues arginine (R) to negatively charged amino acid is also important because this is necessary to condense the payload into nanoparticles through electrostatic interactions. It is generally accepted that a nanoparticle less than <200 nm will be small enough to cross the cell membrane. In addition, the ratio of positively charged residues ensures an overall positively charged nanoparticle which has two main advantages. First, that the particles will not aggregate and repel each other, which allows for improved ease of handling and formulation of the peptides and nanoparticles comprising the peptides complexed with nucleic acids. Secondly, as the cell membrane is negatively charged, nanoparticles that are either neutral or mildly positively charged will not enter the cell.

Finally, the peptide has a greater proportion of hydrophobic residues than hydrophilic residues (see Table 1 below) because this enables an amphipathic helical conformation and when the pH lowers in the endosome it is likely that RALA undergoes a conformational change to a mixture of alpha helix and random coil. This conformational change exposes the hydrophobic residues that can then fuse and destabilize the endosomal membrane enabling release to the cytosol. Having more hydrophobic residues increases the extent of membrane destabilisation.

TABLE 1
Properties of amino acids in RALA peptides

| | pH 7 | Polar/Non-Polar | Hydrophilic | Hydrophobic |
|---|---|---|---|---|
| R - Arg | Positive | Polar | Yes | |
| W - Trp | | Non-Polar | | Yes - very |
| E - Glu | Negative | Polar | Yes | |
| L- Leu | | Non-Polar | | Yes - very |
| A - Ala | | Non-Polar | | Yes - mildly |
| H - His | Positive | Polar | Yes | |
| C - Cys | Slightly | Partially Polar | | Yes - mildly |
| | Negative | | | |

As demonstrated in the Examples, such peptides (exemplified using SEQ ID NO: 1) used according to the present invention have improved activity compared to, for example, polyethylenimine (PEI) for nucleic acid delivery in the production of viral vectors. Advantageously, the peptide used according to the invention are less toxic than PEI or other conventional transfection reagent such as, for example, Lipofectamine 2000®.

Preferably, the arginine (R) residues are evenly distributed at every third and/or fourth amino acid position along the entire length of the peptide.

The amount of hydrophilic amino acid residues in the peptide preferably should not exceed approximately 40% or 37% and the ratio of hydrophilic amino acid residues to hydrophobic amino acid residues ratio at pH 7 is from 30:67 to 40:60, preferably 30:70 to 37:63.

Typically the peptide comprises less than approximately 40 amino acid residues. Optionally, the peptide comprises 35, 34, 33, 32, 31, 30 amino acid residues, preferably 30, 29, 28, 27, 26, 25, 24 or 23 amino acid residues.

Ideally, the peptide comprises the consensus sequence EARLARALARALAR.

Optionally, the peptide may comprise the consensus sequences EARLARALARALAR and/or LARALARALRA as highlighted in the preferred sequences according to the invention listed below:

(SEQ ID No: 1)
WEARLARALARALARHLARALARALRACEA
(SEQ ID No: 2)
WEARLARALARALARLARALARALRACEA
(SEQ ID No: 3)
WEARLARALARALARLARALARALRACEA
(SEQ ID No: 4)
WEARLARALARALARELARALARALRACEA
(SEQ ID No: 5)
REARLARALARALARLARALARALRACEA
(SEQ ID No: 6)
REARLARALARALARLARALARALRAREA
(SEQ ID NO: 7)
REARLARALARALARELARALARALRAREA

Ideally, the present invention discloses a peptide comprising the amino acid sequence

X-EARLARALARALAR-Y-LARALARALRA-Z-EA

or a sequence at least 99%, 98%, 97%, 96%, 95%, 94%, 93%, 92%, 91%, 90%, 85%, 80% identical, wherein
X is W or R;
Y is optional and if present is selected from H or E; and
Z is C or R.

The peptide may comprise or consist of one of SEQ ID NOs: 1, 2, 3, 5, 6 or 7, or a fragment thereof. The peptide may comprise or consist of WEARLARALARALARELARALARALRACEA (SEQ ID No. 4). Ideally, the fragment comprises at least 23 amino acids from SEQ ID Nos: 1 to 7.

Table 2 below provides further details of the several different examples amino acid sequences of preferred amphipathic cell penetrating peptides listed above.

TABLE 2
Examples of preferred RALA peptides of the invention

| | | | % Ratio of | |
|---|---|---|---|---|
| | | | Hydrophilic: | +/− charged |
| | SEQ ID | | Neutral amino | amino acid |
| Amino Acid Residue Sequence | NO. | Length | acid residues | residues |
| WEARLARALARALARHLARALARALRACEA | 1 | | | |
| H removed | 2 | 29 mer | 31:70 | 7:2 |
| WEARLARALARALARLARALARALRACEA | | | | |
| H replaced with glutamic acid (E) | 4 | 30 mer | 33:67 | 7:3 |
| WEARLARALARALARELARALARALRACEA | | | | |
| H removed and W replaced with R | 5 | 29 mer | 34:66 | 8:2 |
| REARLARALARALARLARALARALRACEA | | | | |
| H removed, W replaced with R and C | 6 | 29 mer | 37:63 | 9:2 |
| replaced with R | | | | |
| REARLARALARALARLARALARALRAREA | | | | |
| H replaced with E, W replaced with R | 7 | 30 mer | 40:60 | 9:3 |
| and C replaced with R | | | | |
| REARLARALARALARELARALARALRAREA | | | | |
| Comparative peptide | 8 | 22 mer | | |
| WEARLARALARALARHLRACEA | | | | |

A most preferred sequence comprises/consists of the amino acid sequence WEARLARALARALARHLARALARALRACEA (SEQ ID No: 1), which is exemplified herein. All references herein to amphipathic cell penetrating peptides in the context of the invention may be taken to refer to SEQ ID NO: 1 in particularly preferred embodiments.

It will be understood, in this specification “RALA” is a generic term referring to the RALA sequence (SEQ ID No: 1) or other similar sequences, including but not limited to SEQ ID Nos: 2 to 7, which also fall within the scope of the invention.

The disclosure also encompasses peptides having at least 80%, 85%, 90%, 95%, 96%, 97%, 98% or 99% sequence identity or sequence homology with SEQ ID Nos: 1 to 7, or fragments thereof. Particularly preferred is a peptide which comprises or consists of a peptide with at least 80% sequence identity to SEQ ID NO: 1, or a fragment thereof

Advantageously, the amphipathic cell penetrating peptides used according to the invention consist of arginine/alanine/leucine/alanine repeats that result in a specifically tailored hydrophobic and hydrophilic region facilitating interaction with the lipid bilayers enabling transport of the peptide across cellular membranes. As stated above, the presence of arginine (R) residues is an essential feature of these peptide. There are two main advantages of using arginine. Firstly, arginine has consistently been shown to be the optimal amino acid for condensing DNA with arginine rich sequences binding in milliseconds. Secondly, arginine rich sequences based on the Rev sequence have the capacity to actively transport DNA into the nucleus of cells via the importin pathway.

In addition, there must be at least 2, but no more than 3, glutamate residues (E) to ensure pH-dependent solubility and protonation which facilitates endosomal disruption.

Alternatively, the peptide of the invention may comprise a cell targeting motif sequence conjugated to the N-terminus of the peptide via a spacer sequence. The spacer sequence may be an alpha helical spacer.

Advantageously, we have found that the amphipathic cell penetrating peptide (RALA and similar sequences) or modified peptide/peptide derivatives used according to the present invention facilitate nuclear localisation. This gives these amphipathic cell penetrating peptides a distinct advantage over conventional non-viral and viral delivery systems. These amphipathic cell penetrating peptides form nanoparticles after 5 mins and are stable up to 48 hours at room temperature. The peptides of used according to the invention are typically stable as nanoparticles up to 5, 6 and 15 days after delivery.

The amphipathic cell penetrating peptide can create nanoparticles with a size less than 150 nm or even 100 nm with nucleic acids or other agents. This facilitates transport of these agents across cell membranes, out of the endosomes and to the nucleus. We have found that these nanoparticles are stable in serum and over a temperature range of 4 to 37° C.

The peptides used according to the invention can form nanoparticles comprising the peptide of the invention complexed or condensing with one or more nucleic acid, preferably one or more plasmid. In this specification, it will be understood the terms “complexed” and “condensing” are interchangeable.

Advantageously, the peptide of the invention condenses the one or more nucleic acid (e.g. one or more plasmid). Accordingly, a peptide used in the invention may be complexed with one or more nucleic acid, preferably one or more plasmid, to form discrete spherical nanoparticles, each nanoparticle with a diameter less than approximately 150 nm, preferably less than or equal to 100 nm.

The nanoparticles may have a N:P ratio (the molar ratio of positively charged nitrogen atoms to negatively charged phosphates in DNA). The use of amphipathic cell penetrating peptides to complex with DNA and transfect cells is highly tuneable. The nanoparticle size and charge can be altered by modifying the N:P ratio. Preferably nanoparticles of the invention have an N:P ratio of greater than 2, preferably greater than 4.

### Viral Vectors

The invention relates to the production of viral vectors. In particular, the invention relates to the production of parvoviral vectors, particularly adeno-associated virus (AAV) vectors and/or bocaviral vectors, lentiviral vectors, retroviral vectors, adenoviral vectors, herpes-simplex virus (HSV) vectors, poxvirus vectors and/or baculovirus vectors, and modified versions thereof.

Preferably the invention relates to the production of AAV vectors. AAV are a family of small viruses which infect humans and some other primate species. AAV belong to the genus Dependovirus, of the family Parvoviridae. AAV are small (approximately 20 nm), non-enveloped, replication-deficient viruses. AAV possess a single-stranded linear DNA genomes approximately 4.7 kb in length that may be either positive or negative-sensed.

The AAV genome comprises inverted terminal repeats (ITRs) at both ends of the DNA strand, and two open reading frames (ORFs): rep and cap. The former is composed of four overlapping genes encoding Rep proteins required for the AAV life cycle, and the latter contains overlapping nucleotide sequences of capsid proteins: VP1, VP2 and VP3, which interact together to form a capsid of an icosahedral symmetry.

The genes for the VP1, VP2 and VP3 capsid proteins are generally controlled by a single promoter, designated p40, and all three are translated from a single mRNA. The molecular weights of VP1, VP2 and VP3 are typically about 87, 72 and 62 kiloDaltons (kDa) respectively. The AAV capsid proteins are typically encoded on the AAV DNA genome.

Despite the high seroprevalence of AAV in the human population (approximately 80% of humans are seropositive for AAV2) the virus has not been linked to any human illness. AAV vectors can infect both dividing and quiescent cells. The virus may persist in an extrachromosomal state without integrating into the genome of the host cell. Alternatively, the virus may stably integrate into the host cell genome at a specific site in human chromosome 19 (AAVS1). In contrast to adenoviruses, AAV usually does not trigger an immune response to cells infected with it, and thus can deliver genes to sites of interest, including the brain in the context of gene therapy for diseases of muscle and eye. These features make AAV a very attractive candidate for creating viral vectors for gene therapy, and for developing disease models.

There are currently 11 known AAV serotypes: AAV1, AAV2, AAV3, AAV4, AAV5, AAV6, AAV7, AAV8, AAV10, AAV9, AAV10 and AAV11. Synthetic AAV serotypes, such as hybrid AAV serotypes are also known in the art. The methods of the invention may be used to produce any AAV serotype. Production of AAV2, AAV1 and/or AAV8 is particularly preferred. In addition, are used herein the term “AAV” encompasses modified and synthetic AAV serotypes, such as chimeric or hybrid AAV serotypes. Such modified AAV serotypes may be produced, for example, by capsid engineering.

Adenoviruses are medium-sized (approximately 90-100 nm) non-enveloped double-stranded DNA viruses belonging to the family Adenoviridae. Adenoviruses infect a broad range of vertebrate hosts, including humans. Adenoviral genomes are linear, double-stranded DNA molecules typically about 25 to 50 kilobases (kb) in length.

The term “retrovirus” refers to any member of the Retroviridae family of RNA viruses that encode the enzyme reverse transcriptase. Retroviral genomes are dimers of linear single-stranded, positive-sense RNA molecules, typically from about 7 to about 10 kb in length. Retrovirus virions are enveloped particles of about 100 nm in diameter.

The term “lentivirus” refers to a family of retroviruses. Examples of retroviruses suitable for use in the present invention include gammaretroviruses such as murine leukaemia virus (MLV) and feline leukaemia virus (FLV). Examples of lentiviruses suitable for use in the present invention include Human immunodeficiency virus (HIV), Feline immunodeficiency virus (FIV), Simian immunodeficiency virus (SIV), Equine infectious anaemia virus (EIAV), and Visna/maedi virus. Modified or pseudotyped lentiviral vectors are also encompassed.

Herpes-simplex viruses are enveloped viruses belonging to the family Herpesviridae, composed of a linear, double-stranded DNA genome, with a wide host range. Examples include herpes-simplex viruses 1 and 2 (HSV-1 and -2), varicella zoster virus, Epstein-Barr virus (EBV) and human cytomegalovirus (CMV). Again, modified HSV vectors are also encompassed.

Baculoviruses are enveloped viruses of the family Baculoviridae, and infect a wide range of insect hosts. Baculoviruses contain a circular double-stranded DNA genome typically from 80 to 180 kb in size. Examples of baculoviruses include Autographa californica multicapsid nucleopolyhedrovirus (AcMNPV), Helicoverpa zea nuclear polyhedrosis virus and Spodoptera exigua nuclear polyhedrosis virus. Again, modified baculovirus vectors are also encompassed.

A viral vector produced according to the invention may be integrase-competent (IC). Alternatively, the vector may be integrase-deficient (ID). When a viral vector of the invention is IC, it may comprise terminal repeats either side of the transgene which mediate stable, site-specific integration into the genome of a target cell.

The viral vectors of the present invention may comprise a transgene (e.g. a gene encoding a therapeutic protein) which can be expressed in a target cell upon transduction with the vector. Alternatively, the viral vectors may comprise a therapeutic nucleic acid molecule, such as an mRNA or miRNA. The therapeutic nucleic acid molecule may not encode a protein (such as in the case for miRNA), but exert its therapeutic effect in another way, for example by decreasing or ablating expression of a dysfunctional protein or a protein expressed at pathological levels within the cells of a patient. A viral vector of the invention may be a vaccine vector. Thus, the vector may comprise a nucleic acid encoding for an antigen which will elicit a protective immune response in a patient. A viral vector of the invention may be an oncolytic viral vector. Thus, the vector may either be a wild-type oncolytic virus comprising the wild-type virus genome, or a modified oncolytic virus which comprises an oncolytic virus genome that has been attenuation or modified to increase tumour targeting (e.g. by modifying the gene(s) encoding the viral coat proteins, by placing one or more viral genes under the control of a tumour-specific promoter, or by introducing miRNA response elements).

The viral vectors of the invention may enable high levels of transgene expression, resulting in high levels (therapeutic levels) of expression of a therapeutic protein or nucleic acid. The terms high expression and therapeutic expression are used interchangeably herein. Expression may be measured by any appropriate method (qualitative or quantitative, preferably quantitative), and concentrations given in any appropriate unit of measurement, for example ng/ml or nM.

Expression of a transgene of interest may be given relative to a suitable control, e.g. compared with transduction of an empty vector or a mock transduction, or relative to the expression of a corresponding endogenous (defective) gene in a patient. Expression may be measured in terms of mRNA or protein expression.

The transgene included in the vector of the invention may be modified to facilitate expression. For example, the transgene sequence may be in CpG-depleted (or CpG-free) and/or codon-optimised form to facilitate gene expression. Standard techniques for modifying the transgene sequence in this way are known in the art.

The viral vector typically comprises a promoter operably linked to the transgene, enabling expression of the transgene. Hence, the promoter sequence can be at either or both ends of the transgene. Furthermore, more than one promoter and transgene can be present in one viral vector. Accordingly, more than one transgene can be expressed by one vector.

Promoters for transgene expression are known in the art and their suitability for the viral vectors of the invention determined using routine techniques known in the art. Any promoter may be used in a viral vector of the invention, provided the promoter is capable of driving expression of the transgene when they are operably linked. Such promoters are known in the art, including the AAV E1 promoter or E4 promoter, for example, as well as others including, but not limited to, the CMV promoter, the PGK promoter, UbC and UCOE. The promoter may be tissue or cell preferred or specific, meaning that it drives expression of the transgene in either a particular tissue or cell type of interest. Again, such promoters are known in the art. The promoter may be modified to further regulate expression of the transgene of the invention.

Suitable polyadenylation signals at the 3′ end of the therapeutic polypeptide include, but are not limited to, AAV polyadenylation signals. For AAV vectors, the E3 region of the AAV genome may be deleted in order to increase the cloning capacity of a vector, or it may be left in the vector construct.

The vectors of the invention may be modified to allow shut down of gene expression. Standard techniques for modifying the vector in this way are known in the art. As a non-limiting example, Tet-responsive promoters are widely used.

Viral vectors produced according to the present invention may be used in a range of applications, including gene therapy, vaccine, oncolytic virus therapy, as well as for diagnostics purposes. Viral vectors according to the invention may also be used as research reagents, for example in the context of drug discovery, or for synthesis of biological products, by introducing a transgene into producer cells to elicit recombinant protein production. As used herein, the term “viral vector” encompasses virus particles produced according to the methods of the invention, and which may have the potential to be used in one or more application as described herein.

The methods of the present invention allow for the production of viral vectors such as the viral vectors described above. In particular, the methods of the invention allow for the production of populations of viral vectors, such as populations of the viral vectors as described above, particularly populations of AAV vectors (e.g. AAV2 or AAV8) and/or populations of lentiviral vectors. Accordingly, the invention provides a population of viral vectors obtained by or obtainable by a method of the invention.

Populations of viral vectors produced using the methods of the invention are distinguishable from populations of the same viral vectors made by conventional production methods, and may possess unexpected advantages over such conventionally produced vector populations. In particular, as described in more detail below and as demonstrated in the examples herein, methods of viral vector production using RALA peptides according to the invention significantly increase the ratio of full:empty viral particles (i.e. decrease the empty:full ratio) compared with conventional methods (e.g. using PEI). Accordingly, the invention provides a population of viral vectors, particularly populations of AAV vectors (e.g. AAV2 or AAV8), wherein the ratio of empty:full viral particles is about 24:1 or less, for example about 20:1 or less, about 15:1 or less, about 10:1 or less, about 9:1 or less, about 8:1 or less, about 7:1 or less, about 6:1 or less, about 5:1 or less, about 4:1 or less, about 3:1 or less, about 2:1 or less, or about 1:1. A population of viral vectors according to the invention, particularly a population of AAV vectors (e.g. AAV2 or AAV8), may comprise at least about 10% full vectors, for example at least about 15% full vectors. For example, a population of viral vectors according to the invention may comprise from about 5% full vectors to about 50% full vectors, from about 10% full vectors to about 50% full vectors, such as from about 10% full vectors to about 35% full vectors, or from about 10% full vectors to about 20% full vectors. For transgenes which are cytotoxic to the producer cell, even the production of 5% full vectors would be advantageous, as such toxic transgenes typically result in low yields of full vectors.

The population of viral vectors according to the invention may be produced by methods of the invention, and achieve the improved full particle capacity (defined in terms of empty:full viral particle ratio and/or % full particles), without a step of further isolating, purifying or polishing the viral vectors.

### Methods of Production

The present inventors are the first to demonstrate that amphipathic cell penetrating peptides as described herein are able to be used in the production of viral vectors. Accordingly, the invention provides the use of such peptides in the production of viral vectors.

Said use typically comprises the use of the amphipathic cell penetrating peptide to transfect a producer cell or population of producer cells with one or more nucleic acid.

The invention also provides a method of producing a viral vector as described herein, said method comprising a step of transfecting a population of producer cells with one or more nucleic acid, using an amphipathic cell penetrating peptide as described herein. Said method also typically comprises a step of harvesting the viral vectors (or population thereof) produced by the transfected producer cells.

### Nucleic Acids

For the use or method of the invention, one, two three, four, five or more nucleic acids may be used. Each one or more nucleic acid may be independently selected from any appropriate nucleic acid, e.g. DNA or RNA, double-stranded or single-stranded, linear or circular. Typically each of the one or more nucleic acids is a DNA molecule, such as a plasmid, a closed linear DNA molecule (also known in the art as “doggy bone” DNA or dbDNA) or an episome. Preferably each of the one or more nucleic acids is a plasmid. The term “plasmid” as used herein encompasses nanoplasmids, which typically have a backbone of 500 bp or less.

Said one or more nucleic acid typically comprise the necessary elements to drive viral vector production in the host cell. The elements needed for viral vector production may vary depending on the viral vector to be produced, but typically require one or more nucleic acid (e.g. plasmid) encoding the genome for the viral vector and a nucleic acid (e.g. plasmid) comprising the transgene of interest. One or more helper nucleic acid (e.g. helper plasmid) comprising additional genes needed for productive viral replication may also be used. Therefore, in some embodiments two or three nucleic acids, typically plasmids may be used according to the invention. Standard production systems/nucleic acids (e.g. plasmids) for the production of different viral vector as known in the art and can be readily applied to the methods of the present invention by one of ordinary kill in the art. The method of the invention is not limited to any specific set of production nucleic acids (e.g. plasmids), but rather is platform agnostic with regards to the specific nucleic acids (e.g. plasmids) used.

By way of non-limiting example, a standard protocol for AAV vector production may use: (i) a nucleic acid (e.g. plasmid) comprising a transgene of interest; (ii) a nucleic acid (e.g. plasmid) comprising the AAV Rep and Capsid (RepCap) genes; and (iii) a helper nucleic acid (e.g. plasmid). The helper nucleic acid (e.g. plasmid) typically provides helper genes isolated from adenovirus (e.g. one or more of E1A, E1B, E2A, E40RF6 and VA). Alternatively, rather than a helper plasmid, an AAV helper virus (e.g. an adenovirus or HSV virus) may be used to supply the additional genes needed for productive AAV replication. In which case, the amphipathic cell penetrating peptides are used in connection with the nucleic acid (e.g. plasmid) comprising the transgene and the nucleic acid (e.g. plasmid) comprising the RepCap genes.

Accordingly, when the method or use of the invention relates to the production of AAV vectors, the one or more nucleic acids are plasmids, and optionally said plasmids comprise (i) a plasmid comprising the RepCap genes (a RepCap plasmid); (ii) a plasmid comprising a transgene of interest (a transgene plasmid); and (iii) a helper plasmid. Standard RepCap plasmids, helper plasmids and transgene plasmids for AAV vector production are known in the art and readily available, such as pAdDeltaF6 (helper plasmid), pAAV1/2, pAAV2/2, pAAV2/8, 7M8 (RepCap plasmids) and pUCmini-iCAP-PHP.eB and AAV-CMV-GFP (transgene plasmids). Standard ratios of transgene plasmid: RepCap plasmid: helper plasmid are known in the art, and it is within the routine practice of one of ordinary skill in the art to select and/or optimise the appropriate plasmid ratio for any given AAV serotype and transgene. By way of non-limiting example, a transgene plasmid: RepCap plasmid: helper plasmid molar ratio of 1:1:1 (normalised for plasmid size) is standard in the art (as described in Kimura et al. (Scientific Reports (2019) 9, Article number 13601) or at https://www.addgene.org/protocols/aav-production-hek293-cells/).

By way of a further limiting example, a standard protocol for lentiviral/retroviral production may use: (i) a nucleic acid (e.g. plasmid) comprising the transgene of interest; (i) one or two packaging nucleic acid(s) (e.g. plasmid(s)); and (iii) one or more nucleic acid(s) (e.g. plasmid(s)) encoding one or more envelope proteins. When one packaging nucleic acid (e.g. plasmid) is used, this will typically encode the Gag, Pol, Rev genes and optionally Tat. When two packaging nucleic acids (e.g. plasmids) are used, one will typically encode Rev and one will typically encode Gag and Pol. When two packaging nucleic acids (e.g. plasmids) are used, Tat dependency may be eliminated, such that Tat is no longer comprised in either packaging nucleic acid.

Accordingly, when the method or use of the invention relates to the production of lentiviral/retroviral vectors, the one or more nucleic acids are plasmids, and optionally said plasmids comprise (i) a transgene plasmid (also referred to interchangeably as a transfer plasmid); (ii) a packaging plasmid (comprising Gag, Pol and Rev and optionally Tat); and (iii) a plasmid encoding an envelope protein (an envelope plasmid). Standard transgene plasmids, packaging plasmids and envelope plasmids for lentiviral vector production are known in the art and readily available, such as pRSV-Rev, psPAX2, and pCPRDEnv (packaging plasmids), pCMV-VSVg, RD114 and Cocal (envelope plasmids) and pSico, pLKO.1 puro, FUGW and pLL3.7 (transgene or transfer plasmids). Standard ratios of transgene plasmid: packaging plasmid(s): envelope plasmid for the production of lentiviral/retroviral vectors are known in the art, and it is within the routine practice of one of ordinary skill in the art to select the appropriate plasmid ratio for any given lentiviral/retroviral vector and transgene. By way of non-limiting example, a transgene plasmid: packaging plasmid(s): envelope plasmid molar ratio of 2:2:1 (normalised for plasmid size) is standard in the art (as described in Berger et al. (Nature Protocols (2011) 6(6): 806-816)). The plasmid ratios may be molar ratios or w/w ratios.

Plasmid systems, including exemplary plasmids and suitable plasmid ratios, for other viral vectors are also well-known in the art. For example, in Cosset et al. JOURNAL OF VIROLOGY, Dec. 1995, p. 7430-7436) and Cordeil et al. (J Virol. 2013 March; 87(5): 2587-2596) for retroviruses and Mangeot et al. (Mol. Ther. 2002 March; 5(3):283-90) for SIV. Standard ratios of transgene plasmid: packaging plasmid(s): envelope plasmid molar ratio as described in the art for retroviral vectors and SIV include 2:2:1 (normalised for plasmid size).

The amount of the one or more nucleic acid (e.g. plasmids) used to transfect the producer cells may depend on the viral vector to be produced and/or the producer cell line used. It is within the routine practice of one of ordinary skill in the art to select an appropriate amount of the one or more nucleic acid (e.g. plasmids) for use in a method of the invention. The total amount of the one or more nucleic acids (e.g. plasmids) may be in the region of from about 500 ng to about 20 μg total nucleic acid (e.g. total plasmid) per 10⁶ cells, from about 1 μg to about 20 μg total nucleic acid (e.g. total plasmid) per 10⁶ cells, such as from about 1 μg to about 10 μg total nucleic acid (e.g. total plasmid) per 10⁶ cells, from about 1 μg to about 5 μg total nucleic acid (e.g. total plasmid) per 10⁶ cells, or from about 1 μg to about 2 μg total nucleic acid (e.g. total plasmid) per 10⁶ cells. In some preferred embodiments, the total amount of the one or more nucleic acids (e.g. plasmids) may be from about 1 μg to about 2 μg total nucleic acid (e.g. total plasmid) per 1×10′ cells, particularly for the production of AAV2.

### Nanoparticles

An amphipathic cell penetrating peptide of the invention may be complexed with the one or more nucleic acid (e.g. plasmid) prior to transfection of a producer cell. Typically complexing of the amphipathic cell penetrating peptide and one or more nucleic acid (e.g. plasmid) forms a nanoparticle.

Typically, the amphipathic cell penetrating peptide complexes with the one or more nucleic acid (e.g. plasmid) to form discrete spherical nanoparticles. Each nanoparticle typically has a diameter of less than about 150 nm, preferably less than or equal to 100 nm.

The amphipathic cell penetrating peptide may be complexed with one or more nucleic acid (e.g. plasmid) by contacting the peptide with the one or more nucleic acid. This may produce nanoparticles immediately, or alternatively complexing may comprise incubating the resulting peptide/nucleic acid mixture to facilitate nanoparticle formation. Complexing can be carried out using any appropriate conditions and for any appropriate time, as can be readily determined by one of ordinary skill in the art without undue burden. For example, complexing can be carried out at room temperature (approximately 21° C.), under cold conditions (e.g. 4° C.) or at raised temperatures (e.g. 37° C.). Complexing may be carried out for about 1 minute to about 4 hours, for example about 5-10 minutes, about 15-30 minutes.

The amphipathic cell penetrating peptide may be complexed with one, two, three, four, five or more nucleic acids, such as one, two, three, four, five or more plasmids prior to transfection.

Each nanoparticle may contain a single nucleic acid molecule (e.g. plasmid). Typically each nanoparticle contains multiple nucleic acid molecules (e.g. plasmids). The nucleic acid molecules (e.g. plasmids) within a given nanoparticle may all be the same type of nucleic acid. By way of non-limiting example, a nanoparticle may contain multiple copies of a transgene plasmid, but no copies of any other nucleic acid. Alternatively, a nanoparticle may contain two or more different types of nucleic acid. By way of non-limiting example, a nanoparticle may contain one or more copy of a transgene plasmid and one or more copy of a helper plasmid.

Each of the one or more nucleic acids (e.g. plasmids) may complexed separately with the peptide. In such instances, each nanoparticle will contain a single type of nucleic acid (e.g. plasmid), although optionally multiple copies of said nucleic acid may be present within said nanoparticle.

Alternatively, the one or more nucleic acids (e.g. plasmids) may be pooled prior to complexing with the amphipathic cell penetrating peptide. In such instances, each nanoparticle may contain a single type of nucleic acid (e.g. plasmid), or one or more copy of at least two different nucleic acids (e.g. plasmids).

A single amphipathic cell penetrating peptide may be used according to the invention, or multiple amphipathic cell penetrating peptides may be used. Preferably, if a single amphipathic cell penetrating peptide is used the peptide comprises or consists of SEQ ID NO: SEQ ID NO: 1, 2, 5 or a fragment thereof, particularly SEQ ID NO: 1 or a fragment thereof. When multiple amphipathic cell penetrating peptides are used, these may be used in combination, such that an individual nanoparticle comprises multiple amphipathic cell penetrating peptides. Alternatively, different amphipathic cell penetrating peptides may be used to form nanoparticles with the different nucleic acids, such that each nanoparticle comprise a single type (e.g. sequence) of amphipathic cell penetrating peptide. By way of non-limiting example, nanoparticles for an AAV transgene plasmid may be formed using the peptide of SEQ ID NO: 1, nanoparticles for an AAV RepCap plasmid may be formed using the peptide of SEQ ID NO: 2 and nanoparticles for an AAV helper plasmid may be formed using the peptide of SEQ ID NO: 2 or 5.

The use of amphipathic cell penetrating peptides for the production of viral vectors is advantageous as nanoparticles of an amphipathic cell penetrating peptide complexed with a nucleic acid (e.g. plasmid) are amenable to lyophilisation. The lyophilised nanoparticles are highly stable compared with the nucleic acids (e.g. plasmids) used in viral vector production, which typically require storing at temperatures of −80° C. or less. In contrast, the lyophilised nanoparticles of the invention can be stored at higher temperatures, e.g. room temperature, and can be stored at these temperatures for prolonged periods of time, as described herein.

### Transfecting

As described herein, the amphipathic cell penetrating peptides of the invention are used to produce viral vectors, and methods of producing viral vectors using said amphipathic cell penetrating peptides are provided. Typically the amphipathic cell penetrating peptides are used to transfect producer cells with one or more nucleic acid (e.g. plasmids), such as the nucleic acids described herein. In particular, the amphipathic cell penetrating peptides can be complexed with the one or more nucleic acid (e.g. plasmids) as described herein to form nanoparticles, and the nanoparticles used to transfect producer cells. Typically the transfection is carried out in vitro or ex vivo, with in vitro transfection being preferred.

When the one or more nucleic acids (e.g. plasmids) are pooled prior to complexing with the peptide to form nanoparticles as described herein, the resulting population of nanoparticles can be used to transfect producer cells in a single step.

When separate populations of nanoparticles are produced for each of the one or more nucleic acids (e.g. plasmids) as described herein, these different populations of nanoparticles may be pooled prior to transfection of producer cells. Thus, the pooled nanoparticles may be used to transfect producer cells in a single step.

Alternatively, when separate populations of nanoparticles are produced for each of the one or more nucleic acids (e.g. plasmids) as described herein, the separate populations of nanoparticles may be used to transfect producer cells in a sequential manner. The order in which the populations of nanoparticles are used to transfect producer cells may not be particularly limited, for example to reduce the stress to the producer cells by transfecting with the separate populations of nanoparticles one at a time.

The order in which the populations of nanoparticles are used to transfect producer cells may be selected based on the biochemical properties of one or more of the viral proteins, and/or the protein expressed by a transgene; and/or on the effect of said protein(s) on the producer cell. The order in which the populations of nanoparticles are used to transfect producer cells may be selected to emulate the temporal expression of viral genes that would occur with a wild-type virus Thus, transfection with nanoparticles comprising early genes may occur early in sequence of transfections, and transfections with nanoparticles comprising late genes may occur late in the sequence of transfections.

Alternatively or in addition, the order in which the populations of nanoparticles are used to transfect producer cells may be selected based on factors such as the toxicity of the one or more nucleic acids (e.g. plasmids) to be transfected and/or the producer cell type. It is known in the art that the size and form of nucleic acids can influence their toxicity, particularly the specific ORFs encoded by the nucleic acids can influence toxicity. It is therefore within the routine practice of one of ordinary skill in the art to determine which of the one or more nucleic acids (e.g. plasmids) are more toxic, and then order the transfection of the producer cells on that basis. It may be preferable to transfect the producer cells with the most toxic nucleic acid (e.g. plasmid), or nanoparticles comprising said nucleic acid first, so that the producer cells are at their healthiest and most able to withstand the toxicity. The remaining one or more nucleic acids (e.g. plasmids), or nanoparticles comprising said nucleic acids may then be subsequently used to transfect the producer cells, preferably in descending order of toxicity. Alternatively, it may be preferable to transfect the producer cells with the least toxic nucleic acid (e.g. plasmid), or nanoparticles comprising said nucleic acid first, so that the producer cells are at their healthiest and transfection efficiency is maximised. The remaining one or more nucleic acids (e.g. plasmids), or nanoparticles comprising said nucleic acids may then be subsequently used to transfect the producer cells, preferably in increasing order of toxicity.

When the one or more nucleic acids (e.g. plasmids), or separate populations of nanoparticles comprising each of the one or more nucleic acids (e.g. plasmids) are used to transfect producer cells in a sequential manner, the timing of transfection with each one or more nucleic acids (e.g. plasmids), or separate populations of nanoparticles comprising each of the one or more nucleic acids (e.g. plasmids) may not be particularly limited. The timing of transfection with each one or more nucleic acids (e.g. plasmids), or separate populations of nanoparticles comprising each of the one or more nucleic acids (e.g. plasmids) may be selected to optimise the transfection, for example to improve transfection efficiency and/or to decrease toxicity. The one or more nucleic acids (e.g. plasmids), or separate populations of nanoparticles comprising each of the one or more nucleic acids (e.g. plasmids) may be administered between about 2 hours and about 24 hours apart, for example between about 12 hours and about 24 hours apart, between about 2 hours and about 6 hours apart, about 12 hours apart or about 24 hours apart; or between about 1 day and about 1 week apart, for example between about 1 day and about 4 days apart, between about 1 day and about 2 days apart, between about 5 days and about 1 week apart, about 1 day apart or about 2 days apart.

As exemplified herein, the use of amphipathic cell penetrating peptides to produce viral vectors according to the invention is advantageous compared with conventional vector production protocols for several reasons. In particular the use of amphipathic cell penetrating peptides for transfection of producer cells with nucleic acids, typically in the form of nanoparticles, according to the invention offers numerous advantages over the use of conventional transfection reagents and protocols, such as PEI. Accordingly, the methods and reagents of the invention may be compared with a control method/reagent as described herein.

Transfection using amphipathic cell penetrating peptides according to the invention is typically more efficient than using conventional transfection reagents. Transfection efficiency may be quantified in any appropriate units. For example, the % of cells that are successfully transfected, or the number of transfected cells per unit of nucleic acid (e.g. plasmid) used. Any appropriate method can be used to determine transformation efficiency, with standard methods being known in the art.

The increase in transfection efficiency using amphipathic cell penetrating peptides according to the invention may be relative to (compared with) the transfection efficiency using a control method or transfection reagent, as described herein. Typically the control reagent is PEI.

The efficiency of transfection using an amphipathic cell penetrating peptide according to the invention may be at least about 40%, at least about 50%, at least about 60%, at least about 70%, or more. Preferably the efficiency of transfection using an amphipathic cell penetrating peptide according to the invention is at least about 50%, more preferably at least about 60%, even more preferably at least about 70%.

Transfection using an amphipathic cell penetrating peptide according to the invention may increase transfection efficiency by at least about 1.5 times, at least about 2 times, at least about 2.5 times, at least about 3 times, at least about 3.5 times or more. Preferably transfection using an amphipathic cell penetrating peptide according to the invention increases transfection efficiency by at least about 2 times, more preferably at least about 3 times. Any increase in transfection efficiency may be compared with the transfection efficiency of a control method or reagent as described herein. Typically transfection using an amphipathic cell penetrating peptide according to the invention increases transfection efficiency by at least about 1.5 times, at least about 2 times, at least about 2.5 times, at least about 3 times, at least about 3.5 times or more, preferably at least about 2 times, more preferably at least about 3 times, compared with a corresponding transfection using PEI.

As described herein, the amphipathic cell penetrating peptides used according to the invention are less toxic to producer cells (i.e. less cytotoxic) than conventional transfection reagents such as PEI. Cytotoxicity to the producer cells may be quantified in any appropriate units. For example, the % of live cells, increase in cell number or cell growth rate. Any appropriate method can be used to determine transformation efficiency, with standard methods being known in the art. Non-limiting examples of suitable assays include dye exclusion assays (e.g. trypan blue assay), colorimetric assays (e.g. MTT assay) and luminometric assays (e.g. ATP assay).

The reduction in cytotoxicity using amphipathic cell penetrating peptides according to the invention may be relative to (compared with) the cytotoxicity using a control method or transfection reagent, as described herein. Typically the control reagent is PEI.

The cytotoxicity to the producer cells of transfection using an amphipathic cell penetrating peptide according to the invention may be less than about 20%, less than about 15%, less than about 10%, less than about 9%, less than about 8%, less than about 7%, less than about 6%, less than about 5%, less than about 4%, less than about 3%, less than about 2%, less than about 1%, or less. Thus, the percentage of producer cells that die or are non-viable after transfection using an amphipathic cell penetrating peptide according to the invention may be less than about 20%, less than about 15%, less than about 10%, less than about 9%, less than about 8%, less than about 7%, less than about 6%, less than about 5%, less than about 4%, less than about 3%, less than about 2%, less than about 1%, or less. Preferably the cytotoxicity of transfection using an amphipathic cell penetrating peptide according to the invention is less than about 10%, more preferably less than about 5%, even more preferably less than about 2%.

Transfection using an amphipathic cell penetrating peptide according to the invention may decrease cytotoxicity to the producer cells by at least about 5 times (xs), at least about 10xs, at least about 15xs, at least about 16xs, at least about 17xs, at least about 18xs, at least about 19xs, at least about 20xs, or more. Preferably transfection using an amphipathic cell penetrating peptide according to the invention decreases cytotoxicity to the producer cells by at least about 10xs, preferably at least about 15xs, more preferably at least about 20xs. Any decrease in cytotoxicity may be compared with the cytotoxicity of a control method or reagent as described herein. Typically transfection using an amphipathic cell penetrating peptide according to the invention decreases cytotoxicity to the producer cells by at least about 5xs, at least about 10xs, at least about 15xs, at least about 16xs, at least about 17xs, at least about 18xs, at least about 19xs, at least about 20 times, or more compared with a corresponding transfection using PEI.

As described herein, the amphipathic cell penetrating peptides used according to the invention may increase the number of viral capsids produced compared with the number of viral capsids produced using conventional methods and reagents. The number of viral capsids produced may be referred to interchangeably as the viral vector yield or titre. Quantified in this way, the viral vector yield includes both full (i.e. active) viral particles and empty (i.e. inactive) viral particles. In other words, the viral vector yield is the total number of viral particles. The number of viral capsids may be quantified in any appropriate units, for example total particles/mL (TP/mL). Any appropriate method can be used to determine the number of viral capsids, with standard methods being known in the art. Non-limiting examples of suitable assays include ELISA to detect the capsid protein of the particular viral vector being produced.

The increase in the number of viral capsids, i.e. the increase in viral vector yield using amphipathic cell penetrating peptides according to the invention may be relative to (compared with) the number of viral capsids (i.e. viral vector yield) using a control method or transfection reagent, as described herein. Typically the control reagent is PEI.

The number of viral vector capsids produced by transfection using an amphipathic cell penetrating peptide according to the invention may be at least about 1×10⁷ TP/mL, at least about 1×10⁸ TP/mL, at least about 1×10⁹ TP/mL, at least about 2×10⁹ TP/mL, at least about 3×10⁹ TP/mL, at least about 4×10⁹ TP/mL, at least about 5×10⁹ TP/mL, at least about 6×10⁹ TP/mL, at least about 7×10⁹ TP/mL, at least about 8×10⁹ TP/mL, at least about 9×10⁹ TP/mL, at least about 1×10¹⁰ TP/mL, at least about 1.1×10¹⁰ TP/mL, at least about 1.2×10¹⁰ TP/mL, at least about 1.3×10¹⁰ TP/mL, at least about 1.4×10¹⁰ TP/mL, at least about 1.5×10¹⁰ TP/mL, at least about 1.6×10¹⁰ TP/mL, at least about 1.7×10¹⁰ TP/mL, at least about 1.8×10¹⁰ TP/mL, at least about 1.9×10¹⁰ TP/mL, at least about 2×10¹⁰ TP/mL, at least about 3×10¹⁰ TP/mL, at least about 4×10¹⁰ TP/mL, at least about 5×10¹⁰ TP/mL, at least about 1×10¹¹ TP/mL, at least about 5×10¹¹ TP/mL, at least about 1×10¹² TP/mL, or more. Preferably the number of viral vector capsids produced by transfection using an amphipathic cell penetrating peptide according to the invention is at least about 1.0×10⁹ TP/mL, more preferably at least about 1.0×10¹⁰ TP/mL, even more preferably at least about 1.1×10¹⁰, even more preferably at least about 1.5×10¹⁰ TP/mL. By way of non-limiting example, the number of AAV2 capsids produced may be at least about 4×10⁹ TP/mL, at least about, preferably at least about 1×10¹⁰ TP/mL, more preferably at least about 2×10¹⁰ TP/mL. By way of further non-limiting example, the number of AAV8 capsids produced may be at least about 1×10⁹ TP/mL, preferably at least about 9×10⁹ TP/mL, more preferably at least about 1×10¹⁰ TP/mL, even more preferably at least about 1×10¹¹ TP/mL.

Transfection using an amphipathic cell penetrating peptide according to the invention may increase the number of viral vector capsids produced by at least about 1.5 times (xs), at least about 2xs, at least about 2.5xs, at least about 3xs, at least about 5xs, at least about 10xs, at least about 15xs or more. Preferably transfection using an amphipathic cell penetrating peptide according to the invention increases the number of viral vector capsids produced by 2xs, more preferably at least about 3xs. Any increase in the number of viral vector capsids produced may be compared with the number of viral vector capsids produced by a control method or reagent as described herein. Typically transfection using an amphipathic cell penetrating peptide according to the invention increases the number of viral vector capsids produced by at least about 1.5xs, at least about 2xs, at least about 2.5xs, at least about 3xs or more, compared with a corresponding transfection using PEI.

Transfection using an amphipathic cell penetrating peptide according to the invention may increase the number of viral vector capsids produced for at least 2 days, at least 3 days, at least 4 days, at least 5 days, at least 7 days, or more. Preferably, transfection using an amphipathic cell penetrating peptide according to the invention may increase the number of viral vector capsids produced for at least 4 days, preferably for at least 6 days. Again, the duration of the increased viral titre may be compared with a control, as described herein.

Transfection using an amphipathic cell penetrating peptide according to the invention may increase the number of viral vector capsids produced for at least one, at least two, at least three, at least four, at least five, at least ten, at least 20, at least 30, at least 40 or more passages of the producer cells in culture.

Alternatively, transfection using an amphipathic cell penetrating peptide according to the invention may increase the number of viral vector capsids produced indefinitely.

As exemplified herein, the amphipathic cell penetrating peptides used according to the invention may increase the number of viral vector genomes (also referred to interchangeably herein as viral genomes) produced compared with the number of viral vector genomes produced using conventional methods and reagents. The number of viral vector genomes may be quantified in any appropriate units, for example viral vector genomes/mL (VG/mL). Any appropriate method can be used to determine the number of viral vector genomes, with standard methods being known in the art. Non-limiting examples of suitable assays include qPCR to quantify the genome of the particular viral vector being produced.

The increase in the number of viral vector genomes using amphipathic cell penetrating peptides according to the invention may be relative to (compared with) the number of viral vector genomes using a control method or transfection reagent, as described herein. Typically the control reagent is PEI.

The number of viral vector genomes produced by transfection using an amphipathic cell penetrating peptide according to the invention may be at least about 6×10⁵ VG/mL, at least about 1×10⁶ VG/mL, at least about 2×10⁸ VG/mL, at least about 3×10⁸ VG/mL, at least about 4×10⁸ VG/mL, at least about 5×10⁸ VG/mL, at least about 6×10⁸ VG/mL, at least about 7×10⁸ VG/mL, at least about 8×10⁸ VG/mL, at least about 9×10⁸ VG/mL, at least about 1×10⁹ VG/mL, at least about 2×10⁹ VG/mL, at least about 5×10⁹ VG/mL, at least about 1×10¹⁰ VG/mL, at least about 5×10¹⁰ VG/mL, at least about 1×10¹¹ VG/mL, at least about 5×10¹¹ VG/mL, at least about 1×10¹² VG/mL, or more. Preferably the number of viral vector genomes produced by transfection using an amphipathic cell penetrating peptide according to the invention is at least about 1×10⁸ VG/mL, more preferably at least about 2×10⁸ VG/mL, even more preferably at least about 5×10⁸ VG/mL even more preferably at least about 1×10⁹ VG/mL, even more preferably at least about 1×10¹⁰ VG/mL, even more preferably at least about 1×10¹¹ VG/mL. By way of non-limiting example, the number of AAV2 capsids produced may be at least about 5×10⁸ VG/mL, at least about, preferably at least about 8×10⁸ VG/mL, more preferably at least about 1×10⁹ VG/mL. By way of further non-limiting example, the number of AAV8 capsids produced may be at least about 3×10⁸ VG/mL, preferably at least about 7×10⁸ VG/mL, more preferably at least about 1×10⁹ VG/mL.

Transfection using an amphipathic cell penetrating peptide according to the invention may increase the number of viral vector genomes produced by at least about 1.5xs, at least about 2xs, at least about 2.5xs, at least about 3xs or more, either in terms of production per producer cell or per unit volume, typically in terms of production per unit volume (e.g. production per mL). Preferably transfection using an amphipathic cell penetrating peptide according to the invention increases the number of viral vector genomes produced by 2xs, more preferably at least about 3xs. Any increase in the number of viral vector genomes produced may be compared with the number of viral vector genomes produced by a control method or reagent as described herein. Typically transfection using an amphipathic cell penetrating peptide according to the invention increases the number of viral vector genomes produced by at least about 1.5xs, at least about 2xs, at least about 2.5xs, at least about 3xs or more, compared with a corresponding transfection using PEI.

Transfection using an amphipathic cell penetrating peptide according to the invention may increase the number of viral vector genomes produced for at least 2 days, at least 3 days, at least 4 days, at least 5 days, at least 7 days, or more. Preferably, transfection using an amphipathic cell penetrating peptide according to the invention may increase the number of viral vector genomes produced for at least 4 days, preferably for at least 6 days. Again, the duration of the increased viral vector genome production may be compared with a control, as described herein.

Transfection using an amphipathic cell penetrating peptide according to the invention may increase the number of viral vector genomes produced for at least one, at least two, at least three, at least four, at least five, at least ten, at least 20, at least 30, at least 40 or more passages of the producer cells in culture.

Alternatively, transfection using an amphipathic cell penetrating peptide according to the invention may increase the number of viral vector genomes produced indefinitely.

Significantly, as described herein, the amphipathic cell penetrating peptides used according to the invention typically increase the full:empty ratio of viral vector capsids (also referred to interchangeably herein as full viral vectors or full viral vector particles) produced compared with the full:empty viral vector capsids produced using conventional methods and reagents. Put another way, the amphipathic cell penetrating peptides used according to the invention typically decrease the empty:full ratio of viral vector capsids produced compared with the empty:full viral vector capsids produced using conventional methods and reagents. The amphipathic cell penetrating peptides used according to the invention may increase the % of full viral vector capsids produced compared with the % full viral vector capsids produced using conventional methods and reagents.

The relative proportions of full number of viral vector capsids compared with empty viral vector capsids may be quantified in any appropriate units, for example as a ratio of empty:full viral vector capsids, a ratio of full:empty viral vector capsids or the % of full viral vector capsids. Any appropriate method can be used to determine the relative proportions of full number of viral vector capsids compared with empty viral vector capsids, with standard methods being known in the art. As a non-limiting example, the number of viral vector genomes can be calculated as described herein, as can the total number of viral vector capsids (full+empty). These values can be used to calculate full:empty, empty:full or % full viral vector capsids.

The increase in the full:empty capsid ratio or % full viral vector capsids (or the decrease in empty:full viral vector capsids) may be relative to (compared with) the full:empty capsid ratio, % full viral vector capsids, or empty:full viral vector capsids produced using a control method or transfection reagent, as described herein. Typically the control reagent is PEI.

The empty:full ratio of viral vector capsids produced by transfection with an amphipathic cell penetrating peptide according to the invention may be about 30:1 or less, about 25:1 or less, about 24:1 or less, about 20:1 or less, about 15:1 or less, about 10:1 or less, about 8:1 or less, about 6:1 or less, about 2:1 or less, or about 1:1 or less, or about 0.2:1. Preferably the empty:full ratio of viral vector capsids produced by transfection with an amphipathic cell penetrating peptide according to the invention is about 10:1 or less, more preferably about 8:1 or less, even more preferably about 6:1 or less, even more preferably about 2:1 or less.

The % full viral vector capsids produced by transfection with an amphipathic cell penetrating peptide according to the invention may be in the range of about 5% to about 80%. The % full viral vector capsids produced by transfection with an amphipathic cell penetrating peptide according to the invention may be at least about 5%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, at least about 40%, at least about 50%, at least about 60%, at least about 70%, at least about 80%, or more. Preferably the % full viral vector capsids produced by transfection with an amphipathic cell penetrating peptide according to the invention is at least about 12%, more preferably at least about 15%, even more preferably at least about 20%. By way of non-limiting example, the % full viral AAV2 capsids produced may be at least about 12%, preferably at least about 16%, more preferably at least about 20%. By way of further non-limiting example, the % full viral AAV8 capsids produced may be at least at least about 10%, preferably at least about 15%, more preferably at least about 20%.

Transfection with an amphipathic cell penetrating peptide according to the invention may increase the number of full viral vectors (full viral vector capsids) produced by at least about 20%, at least about 30%, at least about 35%, at least about 40%, at least about 45%, at least about 50%, at least about 55%, at least about 60%, at least about 65%, at least about 70%, or more. Preferably transfection with an amphipathic cell penetrating peptide according to the invention may increase the number of full viral vector capsids (full viral vector capsids) produced by at least about 40%, more preferably at least about 40%, even more preferably at least about 50%, even more preferably by at least about 60%.

Any increase the % full viral vector capsids, increase in the full:empty viral vector capsid ratio and/or decrease the empty:full viral vector capsid ratio produced according to the invention may be compared with the % full viral vector capsids, full:empty viral vector capsid ratio and/or empty:full viral vector capsid ratio produced by a control method or reagent as described herein. By way of non-limiting example, transfection with an amphipathic cell penetrating peptide according to the invention may increase the number of full viral vector capsids produced by at least about 20%, at least about 30%, at least about 35%, at least about 40%, at least about 45%, at least about 50%, at least about 55%, at least about 60%, at least about 65%, at least about 70%, or more, compared with a corresponding transfection using PEI.

Transfection with an amphipathic cell penetrating peptide according to the invention may increase the % full viral vector capsids, increase the full:empty viral vector capsid ratio, and/or decrease the empty:full viral vector capsid ratio for at least 2 days, at least 3 days, at least 4 days, at least 5 days, at least 7 days, or more. Preferably, transfection using an amphipathic cell penetrating peptide according to the invention may increase the % full viral vector capsids, increase the full:empty viral vector capsid ratio, and/or decrease the empty:full viral vector capsid ratio for at least 4 days, preferably for at least 6 days. Again, the duration of the increases % full viral vector capsids, increased full:empty viral vector capsid ratio, and/or decreased empty:full viral vector capsid ratio may be compared with a control, as described herein.

Transfection using an amphipathic cell penetrating peptide according to the invention may increase the % full viral vector capsids, increase the full:empty viral vector capsid ratio, and/or decrease the empty:full viral vector capsid ratio for at least one, at least two, at least three, at least four, at least five, at least ten, at least 20, at least 30, at least 40 or more passages of the producer cells in culture.

Alternatively, transfection using an amphipathic cell penetrating peptide according to the invention may increase the % full viral vector capsids, increase the full:empty viral vector capsid ratio, and/or decrease the empty:full viral vector capsid ratio indefinitely.

The advantages of the present invention as described herein, including: increased transfection efficiency; decreased cytotoxicity to the producer cells; increased number of viral vector capsids produced; increased number of viral vector genomes produced; and/or increased % full viral vector capsids, increased full:empty viral vector capsid ratio, and/or decreased empty:full viral vector capsid ratio; may be obtained in combination or independently.

Typically, decreased cytotoxicity to the producer cells and/or increased transfection efficiency, preferably both decreased cytotoxicity to the producer cells and increased transfection efficiency are obtained in addition to one or more of the other advantages as described herein. By way of non-limiting example, decreased cytotoxicity to the producer cells and/or increased transfection efficiency, preferably both decreased cytotoxicity to the producer cells and increased transfection efficiency, may be obtained in addition to increased % full viral vector capsids, increased full:empty viral vector capsid ratio, and/or decreased empty:full viral vector capsid ratio.

The increase in % full viral vector capsids, increase in full:empty viral vector capsid ratio and/or decrease in empty:full viral vector capsid ratio produced according to the invention may obtained in addition to an increase in the number of viral vector capsids produced and/or an increase in the number of viral vector genomes produced. Alternatively, the increase in % full viral vector capsids, increase in full:empty viral vector capsid ratio and/or decrease in empty:full viral vector capsid ratio produced according to the invention may obtained independently or without a corresponding increase in the number of viral vector capsids produced and/or increase in the number of viral vector genomes produced. Again, these increases/decreases may be relative to the corresponding values obtained using a control method or transfection reagent, as described herein. Typically the control reagent is PEI.

Although the Examples herein focus on the advantages of using amphipathic cell penetrating peptides in the production of viral vectors, the invention relates more generally to the use of amphipathic cell penetrating peptides in the production of advanced therapy medicinal products (ATMPs), wherein the production methods comprise a transfection step. This is because the advantages demonstrated in the Examples, including decreased cytotoxicity, increased transfection efficiency and/or increased transgene expression (resulting in increased protein production) would also be achieved during transfection for the production of other ATMPs. By way of non-limiting example, the production of cell therapy or diagnostic products which comprise transfecting the cells with a transgene of interest.

### Culture Systems and Methods

The methods of the invention may be carried out using any appropriate cell culture conditions and culture media. Suitable cell culture systems, conditions and media are well-known in the art and it is within the routine practice of one of ordinary skill in the art to select an appropriate cell culture system, cell culture conditions and/or cell culture media for any given producer cell line.

Exemplary, non-limiting culture conditions for producer cells typically include culturing at 33 to 39° C., and preferably around 37° C. The CO₂ concentration may be between about 0% to about 10%, preferably about 5%. The O₂ concentration may be between about 10% to about 30%, preferably about 20%.

Non-limiting examples of cell culture media that can be used for the culture of producer cells include Dulbecco's MEM, OptiMEM, IMDM and RPMI-1640 that can be supplemented with a variety of different nutrients, growth factors, cytokines, etc. The media can be serum free or supplemented with suitable amounts of serum such as fetal bovine serum (FBS) or human serum. The producer cells may be grown in a media which contains human components. Xeno-free or defined culture media (comprising or consisting of synthetically produced components) may also be used according to the invention.

The methods of the invention may comprise a step of culturing the producer cells prior to transfection. By way of non-limiting example, the producer cells may be grown for about 1, 2, 3, 4, 5, 6, 7, 8, 9, 10, 11, 12, 13, 14, 15, 16, 17, 18, 19, 20, or 21 days prior to transfection. Such a culture step may be included in a method of the invention to allow the producer cells to equilibrate with the culture vessel, or to allow time for recovery if the producer cells have been thawed and reconstituted after freezing.

The methods of the invention may comprise a step of expanding the producer cells prior to transfection. By way of non-limiting example, the producer cells may be expanded for about 1, 2, 3, 4, 5, 6, 7, 8, 9, 10, 11, 12, 13, 14, 15, 16, 17, 18, 19, 20, or 21 days prior to transfection. Such an expansion step may be included in a method of the invention to allow the producer cells to establish within the culture vessel, to provide a significant number of producer cells for transfecting and/or to reach a desired concentration to optimise transfection.

An expansion step may comprise culturing the producer cells under expansion conditions. Expansion conditions may comprise the inclusion of one or more cytokine or growth factor in the culture medium to stimulate proliferation of the producer cells. The specific growth factors or cytokines may depend on the specific producer cell line being used, and it is within the routine practice of one of ordinary skill in the art to select suitable factors. Alternatively or in addition, expansion conditions may comprise culturing the producer cells in high-serum medium. As used herein, the term “high-serum medium” means medium containing a concentration of serum which is optimal for proliferation of the producer cells. For example, high-serum medium typically contains at least about 10% serum (e.g. FBS), such as 10% FBS, or 15% FBS. The concentration of serum in a high-serum medium may depend on the particular producer cell line being used. High-serum media also encompasses the use of media which contain serum replacements, wherein the serum replacements provide equivalent growth-supporting properties. All references herein to serum encompass serum replacements unless explicitly stated to the contrary. Serum replacements are commercially available and can be used instead of serum to comply with GMP requirements, when defined culture conditions are required, and/or when the final viral vector product is intended for human use. It is within the routine practice of one of ordinary skill in the art to select an appropriate concentration of serum (or suitable serum replacement) for a high-serum medium for any given producer cell line.

The starting number of producer cells (e.g. prior to expansion) may be at least about 10³, 10⁴, 10⁵, 10⁶, 10⁷, 10⁸, 10⁹, 10¹⁰, or more cells. The starting producer cell population may have a seeding density of at least or about 10, 10², 10³, 10⁴, 10⁵, 10⁶, 10⁷, 10⁸ cells/mL, or more. Expansion of the producer cells may increase the number of producer cells to at least about 10⁷, 10⁸, 10⁹, 10¹⁰, 10¹¹, 10¹², 10¹³, or more. The expanded producer cell population may have a concentration of at least or about 10⁶, 10⁷, 10⁸, 10⁹, 10¹⁰ cells/mL, or more.

The step(s) of culturing and/or expanding the producer cells may comprise changing the culture medium one or more times. Typically media change(s) may be to replace the culture medium with fresh medium of the same type, for example a high-serum medium is changed for fresh high-serum medium of the same type. Such media changes typically serve to replenish the nutrients depleted by growth of the producer cells. It is within the routine practice of one of ordinary skill in the art to determine whether one or media change is appropriate for any given producer cell line, and to select an appropriate timepoint for the one or more media change. For example, media may be changed every 1 day, every 2 days, every 3 days, every 4 days or more during the culture and/or expansion of the producer cells.

The methods of the invention may comprise a combined culture and expansion step. The culture conditions and medium used to culture the producer cells prior to transfection may also facilitate expansion of the producer cells. Thus, any disclosure herein in relation to a step of culturing the producer cells applies equally and without reservation to a step of expanding the producer cells, and any disclosure herein in relation to a step of expanding the producer cells applies equally and without reservation to a step of culturing the producer cells.

The methods of the invention may comprise a step of changing the culture medium of the producer cells prior to transfecting the producer cells. The media change may be to replace the culture medium with fresh medium of the same type. Typically, the media change may be to replace the culture medium with medium with a different concentration of serum. Preferably, when a method of the invention comprises a step of culturing and/or expanding the producer cells in a high-serum culture medium prior to transfection, the method may further comprise a step of changing the culture medium to a low-serum or serum-free culture medium prior to transfection. Serum-free medium contains no serum (whether fetal calf serum, human serum or other). As used herein, the term “low-serum medium” means medium containing a lower concentration of serum than that which is optimal for growth of the producer cells. For example, low-serum medium typically contains 5% serum (e.g. FBS) or less, such as 5% FBS, 4% FBS or 2% FBS. The concentration of serum in a low-serum medium may depend on the particular producer cell line and/or culture system being used. By way of non-limiting example, for suspension culture serum-free medium may be used at least during the transfection step, and in some instances serum-free medium is used throughout the suspension culture. It is within the routine practice of one of ordinary skill in the art to select an appropriate concentration of serum for a low-serum medium for any given producer cell line. The step of changing the culture medium of the producer cells prior to transfection may be carried out between about 0 and about 12 hours prior to transfection, preferably between about 0 and about 8 hours prior to transfection, more preferably between about 0 and about 4 hours prior to transfection, even more preferably between about 0 and about 2 hours prior to transfection. In some embodiments, the step of changing the culture medium of the producer cells prior to transfection may be carried out between about 1 and about 3 hours prior to transfection, particularly at about 2 hours prior to transfection.

According to the invention, transfection of the producer cells with the one or more nucleic acids (e.g. plasmids), typically in the form of nanoparticles as described herein, may be carried out in serum-free or low-serum medium. Serum-free medium contains no serum (whether fetal calf serum, human serum or other). As used herein, the term “low-serum medium” means medium containing a lower concentration of serum than that which is optimal for growth of the producer cells. For example, low-serum medium typically contains 5% serum (e.g. FBS) or less, such as 5% FBS, 4% FBS or 2% FBS. The concentration of serum in a low-serum medium may depend on the particular producer cell line being used. It is within the routine practice of one of ordinary skill in the art to select an appropriate concentration of serum for a low-serum medium for any given producer cell line.

The methods of the invention may comprise a step of changing the culture medium of the producer cells after transfecting the producer cells. The media change may be to replace the culture medium with fresh medium of the same type. Typically, the media change may be to replace the culture medium with medium with a different concentration of serum. Preferably, when a method of the invention comprises transfecting the producer cell lines in low-serum culture medium, the method may further comprise a step of changing the culture medium to a high-serum culture medium after transfection. The step of changing the culture medium of the producer cells after transfection may be carried out between about 0 and about 24 hours after transfection, preferably between about 0 and about 12 hours after transfection, more preferably between about 0 and about 8 hours after transfection, even more preferably between about 0 and about 6 hours after transfection. In some embodiments, the step of changing the culture medium of the producer cells after transfection may be carried out between about 1 and about 6 hours after transfection, particularly at about 5 hours after transfection.

The methods of the invention may comprise harvesting the viral vector from the culture medium and/or the transfected producer cells. Harvesting of the viral vectors from the producer cells typically comprise lysis of the producer cells. Lysis of the producer cells can be achieved using routine methods and reagents (e.g. detergents or surfactants) known the art. Non-limiting examples of such lysis agents include Triton X-100, Triton X-114, NP-40, Brij-35, Brij-58, Tween 20, Tween 80, Octyl glucoside, Octyl thioglucoside, SDS, CHAPS and CHAPSO. A cell lysis reagent for use according to the invention may or may not comprise one or more nuclease. If the viral vectors is harvested from the culture medium alone, the producer cells may be left intact. This is particularly preferable for continuous culture systems as described herein. In such instances, no lysis agent is required.

Methods of the invention may comprise a step of purifying the viral vectors. Typically such a step isolates the viral vectors once the vectors have been harvested from the culture medium and/or producer cells. Thus, purification may isolate the viral vectors from components of the culture medium (including components of the original culture medium and/or other proteins or compounds secreted by the producer cells). Purification may isolate the viral vectors from cellular debris. Such debris may be present in the culture medium due to death of producer cells as the method is carried out, and/or following deliberate lysis of the producer cells to facilitate harvesting of the viral vectors.

Any appropriate purification means may be used to purify the viral vectors. Non-limiting examples of suitable purification steps include depth/end filtration, tangential flow filtration (TFF) and chromatography. The purification step typically comprises at least on chromatography step. Non-limiting examples of chromatography steps that may be used in accordance with the invention include mixed-mode size exclusion chromatography (SEC), affinity chromatography, and/or ion (e.g. anion) exchange chromatography. Elution may be carried out with or without the use of a salt gradient (linear or step gradient).

Purification may comprise the isolation of full viral vectors from empty viral vectors. Alternatively, given the higher yield of full viral vectors using methods of the invention compared with conventional methods, it may not be necessary to remove the empty viral vectors to produce a usable vector product. Accordingly, in some embodiments the methods of the invention do not include a step of purifying the full viral vectors from empty viral vectors, or the purification of the full viral vectors may not be designed to facilitate removal of empty viral vectors. The methods of the invention may not comprise a step of polishing the viral vectors. Preferably the methods of the invention do not include an anion exchange polishing step, and/or an ultrafiltration polishing step.

The methods of the invention may include any combination of: a culturing step; an expansion step; a media change prior to transfection; a media change after transfection; and/or isolation and/or purification of the viral vectors, as described herein.

The methods of the invention can be carried out with producer cells cultured in an adherent culture system or a suspension culture system.

Standard adherent and suspension systems for cell culture are known in the art and are commonly used for culturing producer cells and producing biological products such as proteins and viral vectors. Non-limiting examples of adherent culture systems include HYPERFlask® or HYPERStack®, and fixed-bed systems such as CellCube® or iCellis® bioreactors, or hollow-fibre systems such as Quantum®. Any appropriate adherent cell culture system may be used according to the present invention.

Having demonstrated the advantageous properties of using amphipathic cell penetrating peptides compared with conventional transfection reagents (exemplified with PEI) for producing viral vectors in adherent cell culture, the present inventions have further demonstrated that amphipathic cell penetrating peptides advantageously allow for the efficient production of viral vectors using suspension culture. Accordingly, the methods of the invention can also be carried out using suspension cell culture systems. Such suspension culture is typically capable of producing the advantages described herein. Non-limiting examples of suspension culture systems include spinner flasks and stirred tank bioreactors, such as the Ambr15*, Ambr250*, Biostat®A, BiostatB® or Biostat® B-DCU stirred tank bioreactors or larger scale stirred tank bioreactors, and rocking motion systems. As used herein, suspension culture and suspension culture systems encompasses both batch culture and fed-batch culture. In batch cultures, the producer cells are allowed to grow for a fixed duration of time with all media and reagents added at the beginning, whereas in fed-batch cultures medium and/or additional reagents are added at different intervals. The viral vector products are harvested only after the batch is complete.

In addition, the present inventors have further shown that significant viral vector production is released into the culture medium. Combined with the inventors' surprising demonstration that RALA peptides allow for the efficient production of viral vectors in suspension culture, the invention therefore provides for the production of viral vectors using a continuous production system using amphipathic cell penetrating peptides as described herein.

Continuous production methods of the invention may use adherent or suspension cell culture systems as described herein, preferably a suspension cell culture system. In continuous culture, the viral vector products can be removed at regular intervals (e.g. about every 12 hours, 1 day, 2 days, 3 days, 4 days, 5 days, 6 days, 7 days or more). Fresh culture medium and/or other reagents (including producer cells) can be added at regular intervals (e.g. about every 12 hours, 1 day, 2 days, 3 days, 4 days, 5 days, 6 days, 7 days or more). The intervals at which the viral vector products are removed and the medium and/or other reagents are added may be the same. Alternatively, the intervals at which the viral vector products are removed and the medium and/or other reagents are added may be different, and preferably are independently selected, for example to optimise viral vector yield whilst maintaining the viability and productive capacity of the producer cells.

During continuous production methods, the producer cells may be transfected multiple times. This is because the producer cells do not need to be harvested and/or lysed to extract the viral vector product. Each round of transfection may comprise contacting the producer cells with one or more nucleic acids (e.g. plasmids), typically in the form of nanoparticles, as described herein. Thus, each round of transfection may involve transfecting with all the one or more nucleic acids (e.g. plasmids) necessary to produce a viral vector. Alternatively, each round of transfection may involve transfecting with one or more nucleic acids (e.g. plasmids) necessary to produce a viral vector via sequential transfection as described herein. Typically the nucleic acids (e.g. plasmids) are used in the form of nanoparticles as described herein. Transfection may be carried out at regular intervals (e.g. about every 12 hours, 1 day, 2 days, 3 days, 4 days, 5 days, 6 days, 7 days or more) and the reagents (including the one or more nucleic acids, typically in the form of nanoparticles) may be added at regular intervals as described herein. Without being bound by theory, it is believed that such multiple rounds of transfection may take advantage of the spike in viral vector capsid and viral vector genome production observed in the days immediately following transfection using an amphipathic cell penetrating peptide, as exemplified herein. Without being bound by theory, this spike in viral vector capsid and viral vector genome production may result from improved kinetics of entry of the nanoparticles into the producer cells and/or improved kinetics of entry of the one or more nucleic acids (e.g. plasmids) into the nuclei of producer cells, which can elicit faster expression of the genes comprised in the one or more nucleic acids. Thus, transfection may be preferably carried out at intervals of about every 2 days, about every 3 days or about every 4 days.

Typically, the same one or more nucleic acids (e.g. plasmids) or nanoparticles comprising the same may be used to transfect the producer cells in each round of transfection in a continuous production method. By way of non-limiting example, if a pool of nanoparticles comprising an AAV helper plasmid, an transgene plasmid and an AAV2 RepCap plasmid are used to transfect the producer cells in a first round of transfection, nanoparticles comprising the same AAV helper plasmid, transgene plasmid and AAV2 RepCap plasmid may be used for subsequent rounds of transfection. Alternatively, different one or more nucleic acids (e.g. plasmids) or nanoparticles comprising the same may be used to transfect the producer cells in each round of transfection. Byway of non-limiting example, if a pool of nanoparticles comprising an AAV helper plasmid, an transgene plasmid and an AAV2 RepCap plasmid are used to transfect the producer cells in a first round of transfection, nanoparticles comprising the a different AAV helper plasmid, transgene plasmid and AAV8 RepCap plasmid, or nanoparticles comprising plasmids for the production of a lentiviral vector may be used for subsequent rounds of transfection.

### Producer Cell Lines

According to the present invention, a producer cell (or producer cell line) may be defined as a cell or cell line capable of replicating and packaging a viral vector. Producer cells may be referred to interchangeably as packaging cells. Any appropriate producer cell or producer cell line may be used according to the present invention.

Producer cell lines are typically selected for key characteristics such as origin/derivation, doubling time, permissiveness for viral infection and replication to produce high viral titres and to comply with GMP.

A producer cell of the invention is a eukaryotic cell, and typically a mammalian cell or an insect cell.

A producer cell line of the invention may preferably be a human cell, particularly when the viral vectors produced by the present invention is intended for therapy in humans. Non-limiting examples of producer cells suitable for use according to the invention include HEK293, HEK293T, HEK293-F, NIH3T3, HT1080, A549, and HeLa cells. In a preferred embodiment, the producer cell line is the human embryonic kidney (HEK) 293 cell or a HEK293T cell. The HEK293T cell line expresses the SV40 early region under the transcriptional control of the Rous sarcoma virus long terminal repeat promoter.

A producer cell of the invention may be an insect cell. Non-limiting examples of insect cells suitable for use according to the invention include Sf9, Sf21 and S2 cells.

Producer cells may be used according to the invention in adherent or suspension form.

### Kit of Parts

As described above, nanoparticles of an amphipathic cell penetrating peptide complexed with a nucleic acid (e.g. plasmid) are amenable to lyophilisation, and the lyophilised nanoparticles are highly stable and can be stored for prolonged periods of time. Lyophilised nanoparticles of the invention may be stable for at least 2 months, at least three months, at least four months, at least six months, at least nine months, at least 12 months, at least 18 months or more. The lyophilised nanoparticles may be stable at room temperature (e.g. from about 20 to about 25° C., such as about 21° C.), in cold storage (e.g. about 4° C.), or at elevated temperatures (e.g. from about 30 to about 40° C., such as about 37° C.). Preferably lyophilised nanoparticles are stable at room temperature for at least about four months, more preferably at least about six months.

Lyophilised nanoparticles can therefore be prepared in advance of their use. Lyophilised nanoparticles comprising standard nucleic acids (e.g. plasmids) for use in viral vector production can be prepared in batches and kept in stock ready for use, such that only the nucleic acid encoding sequences to the desired vector (e.g. genome and/or transgene) need to be made as required. By way of non-limiting example, for the production of an AAV vector, a helper plasmid and/or a RepCap may be prepared in advance and lyophilised, either as a combined lyophilised preparation or separate lyophilised preparations for each of the helper plasmid and the RepCap plasmid. The transgene plasmid may be produced as required depending on the specific requirements of the desired AAV vector and transgene. By way of a further non-limiting example, for the production of a lentiviral vector, an envelope plasmid and/or one or more packaging plasmids may be prepared in advance and lyophilised, either as a combined lyophilised preparation or separate lyophilised preparations for each of the envelope plasmid and one or more packaging plasmids. The transgene plasmid may be produced as required depending on the specific requirements of the desired lentiviral vector and transgene.

Accordingly, the present invention provides a kit comprising a composition of lyophilised nanoparticles according to the invention, wherein said lyophilised nanoparticles comprise one or more nucleic acid (e.g. plasmid) for use in the production of a viral vector. By way of non-limiting example, a kit for the production of AAV viral vectors may typically comprise a composition comprising lyophilised nanoparticles comprising a helper plasmid and/or a RepCap plasmid, preferably both a helper plasmid and a RepCap plasmid. By way of a further non-limiting example, a kit for the production of a lentiviral vector may typically comprise a composition comprising lyophilised nanoparticles comprising an envelope plasmid and/or one or more packaging plasmids, preferably both an envelope plasmid and one or more packaging plasmids.

The invention also provides a kit of parts comprising two or more lyophilised nanoparticle compositions, wherein each lyophilised nanoparticle composition comprises lyophilised nanoparticles comprising a single nucleic acid (e.g. plasmid) for use in the production of a viral vector, wherein the two or more lyophilised nanoparticle compositions are separate compositions. The two or more lyophilised nanoparticle compositions may be used simultaneously, separately or sequentially to produce a viral vector, as described herein. By way of non-limiting example, a kit of parts for the production of AAV viral vectors may typically comprise a composition comprising lyophilised nanoparticles comprising a helper plasmid and a separate composition comprising lyophilised nanoparticles comprising a RepCap plasmid. By way of a further non-limiting example, a kit of parts for the production of a lentiviral vector may typically comprise a composition comprising lyophilised nanoparticles comprising an envelope plasmid and a separate composition comprising lyophilised nanoparticles comprising one or more packaging plasmids.

A kit of the invention may optionally further comprise instructions for use, and/or additional reagents for use with the nanoparticle composition(s). Non-limiting examples of addition reagents include buffers for reconstituting the lyophilised nanoparticles, lyophilised amphipathic cell penetrating peptides (which may be used to complex one or more additional nucleic acid (e.g. plasmids) for the production of viral vectors) and/or other lyophilised nanoparticle compositions, e.g. control nanoparticles (such as empty nanoparticles for negative controls), or nanoparticles comprising additional nucleic acids (e.g. plasmids) for the production of viral vectors, such as a transgene plasmid.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

### Alignment Scores for Determining Sequence Identity

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (as described herein) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

### EXAMPLES

The invention is now described with reference to the Examples below. These are not limiting on the scope of the invention, and a person skilled in the art would be appreciate that suitable equivalents could be used within the scope of the present invention. Thus, the Examples may be considered component parts of the invention, and the individual aspects described therein may be considered as disclosed independently, or in any combination.

### Example 1—Transfection of HEK293 Cells in Adherent Culture Using RALA Peptides is Less Cytotoxic to the Cells Compared with Transfection Using PEI

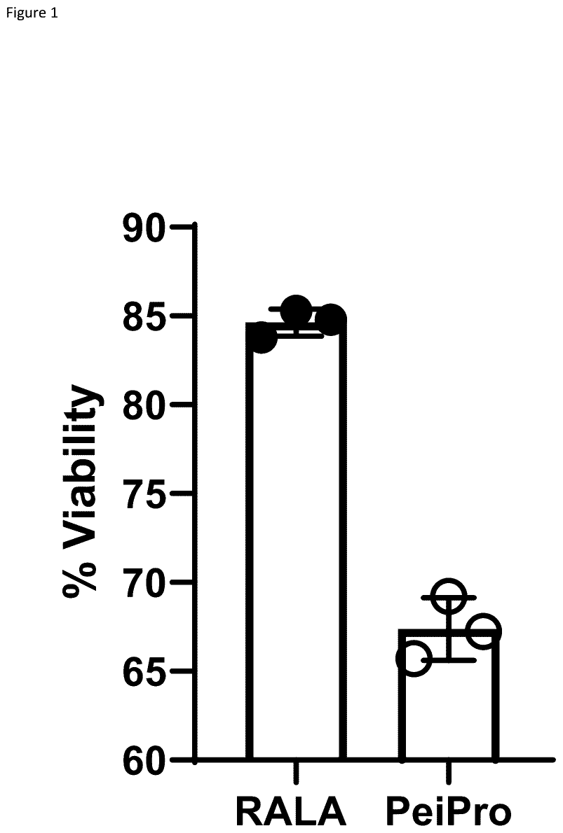

HEK293 cells were seeded at a density of 1×10⁵ cells per well on 24 well plates. 24 hrs later, cells were transfected with 50 ng of plasmid encoding a GFP reporter gene, either complexed with RALA (SEQ ID NO: 1) at N:P10 (14.5 μg of RALA is complexed per 1 μg of DNA) or using PEIpro at a 3:1 ratio. The cells were stained to assess cell viability after 3 days. As can be seen from FIG. 1, the viability of adherent HEK293 cells transfected with plasmid DNA using RALA was significantly greater than the viability of adherent HEK293 cells transfected with plasmid DNA using PEI-Pro.

As such, it was decided to investigate whether using RALA peptides as a transfection agent could improve the production of viral vectors, which require transfection of producer cells with multiple plasmids.

### Example 2—Transfection Using RALA Peptides Increases the Percentage of Full AAV2 Particles Produced in Adherent HEK293 Culture Compared with PEI Transfection

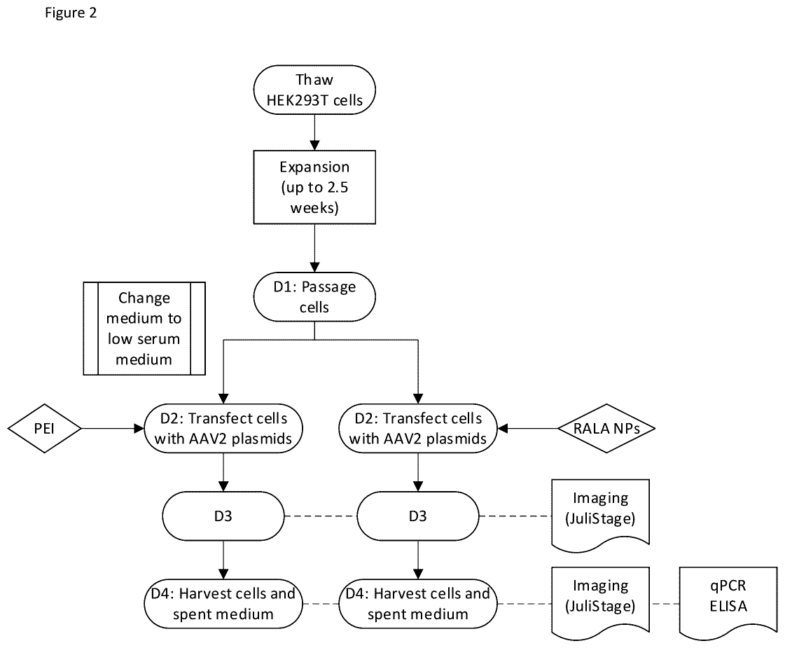

A flow chart of the protocol used is shown in FIG. 2. HEK293T cells were thawed and expanded in DMEM medium containing 10% serum. Following expansion, cells were plated into wells of 6 well plates (with 5% serum containing medium). 24 hrs post plating three wells were harvested to assess cell density.

Nanoparticles were formed by complexing the RepCap, helper and transgene plasmids with RALA peptides of SEQ ID NO: 1. Cells were transfected for 5 hrs with either RALA nanoparticles (N:P10, 14.5 μg of RALA is complexed per 1 μg of DNA) or using PEI-Pro (2:1) as described in Table 3 using 1.56 μg total plasmid per 1×10⁶ cells. The ratio of GFP:RepCap:Helper was kept constant for the different conditions. Non transfected cells were used as a control. 3 wells were used per condition.

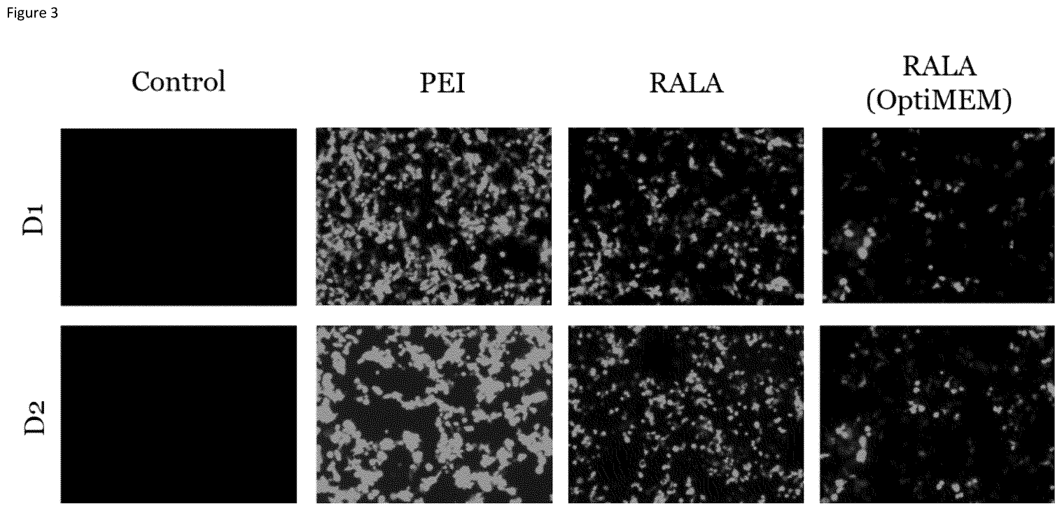

Following transfection, the producer cells were allowed to recover for 48 hrs prior to analysis. Images of cells to assess GFP⁺ expression were taken 24 hrs and 48 hrs post transfection (FIG. 3). 48 hrs post transfection, cells and medium were harvested for assessment of viral production by ELISA and qPCR.

TABLE 3
Details of transfection/NP preparation

| | | PEI-Pro | |
|---|---|---|---|
| RALA nanoparticles | | transfection | No |
| RALA | RALA OptiMEM | PEI | transfection CTRL |
| Transfection in 5% FBS | Medium changed two | Transfection in 10% | Medium was changed |
| No medium exchange | hours prior to | FBS. | to 5% on D 1 but no |
| post transfection | transfection. | Post-transfection | transfection was |
| | Transfection was | medium exchange to | performed. |
| | performed for 5 hrs | 2.5% FBS | |
| | after which OptiMEM | | |
| | was replaced with | | |
| | 10% FBS medium. | | |

qPCR Assay for Viral Genome Quantification

The viral genome titration used was is a Taqman™ based qPCR to quantify ITR2 sequences in the viral genome of AAV2 and AAV2-derived serotypes. The protocol utilised the linearised form of plasmid, serially diluted to generate a standard curve of known quantities of ITR2 sequences. AAV samples were treated with DNase to remove residual DNA, before being lysed at 98° C. to extract the viral genome. The viral genome titre of AAV samples with ITR2 sequences were measured against a standard curve, which was generated using pAAVZSGreen1 plasmid. Physical titre of samples is reported as viral genome per millilitre (vg/mL).

### ELISA Assay for Total Viral Capsid Quantification

The capsid ELISAs are designed to quantify AAV particle number and are based on a sandwich ELISA. For AAV2 capsid quantification, Progen AAV2 ELISA kits were used. A monoclonal antibody specific for a conformational epitope on assembled AAV1 or AAV2 capsids was coated onto strips of a microtiter plate and used to capture AAV2 particles from the specimen, respectively. Standards (kit control) for each serotype were two-fold serially diluted in ASSB buffer to generate a total of 7 dilutions in triplicate for the typical titration curve. The curve allows the quantitative determination of samples of an unknown particle titre.

Samples from each condition were also serially diluted in ASSB buffer to generate a total of three dilutions in triplicate. Both standards and samples were incubated on precoated plates specific for the AAV2 serotype in triplicate. The wells were then incubated with biotinylated detection antibodies followed by an incubation with a Streptavidin peroxidase conjugate. This was followed by addition of 3,3′,5,5′-Tetramethylbenzidine substrate, which reacts to provide the quantifiable colour change. The substrate reaction was stopped using the kit's Stop solution. The optical densities (ODs) of each well were read at 450 nm and 650 nm wavelengths using a plate reader. The 650 nm wavelength served as a reference and the ODs measured were subtracted from the ODs measured at 450 nm to provide the final reading for each well. The blanks ODs were subtracted from all the standard and sample ODs to normalise for any background signal detected. The final ODs of the standards were be used to plot a four-parameter logistic curve in Graphpad Prism and used to interpolate the capsid concentrations of samples. Finally, the interpolated capsid concentrations were multiplied by the relevant dilution factor to calculate the final capsid concentration.

### Results

24 hrs post transfection, cells were images to assess expression of GFP (see FIG. 3). The transfection conditions (cell number, total amount plasmid transfected, etc.) were optimised for PEI, and so PEI-Pro showed a higher proportion of transfected cells than either RALA condition. The RALA condition showed a higher proportion of transfected cells than the RALA OptiMEM condition. The control cells showed no GFP expression. 48 hrs post transfection results show an increase of GFP expression in all conditions. However, the trends seen at 24 hrs remained consistent.

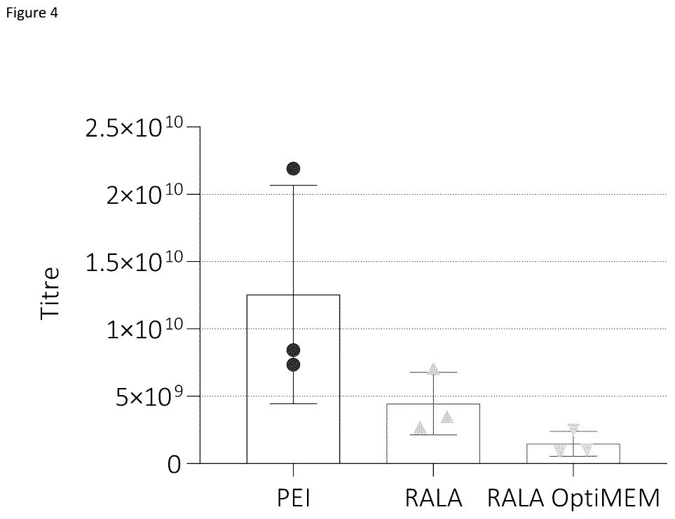

Following imaging, cells and medium were harvested from each well independently to assess viral titre. As described in Table 4 and FIG. 4, viral genome titres as measured by qPCR were lower using RALA compared with the optimised PEI conditions, echoing the results seen on the imaging. Two replicates showed coefficients of variation (CVs) outside the acceptance range (*) and as such were excluded from FIG. 4.

TABLE 4
Viral genome titres as measured by qPCR

| Sample | Titre | CV | N |
|---|---|---|---|
| PEI n = 1 | 2.19E+10 | 13% | 9 |
| PEI n = 2 | 8.43E+09 | 15% | 9 |
| PEI n = 3 | 7.35E+09 | 15% | 9 |
| RALA n = 1 | 3.53E+09 | 20%* | 8 |
| RALA n = 2 | 7.10E+09 | 11% | 9 |
| RALA n = 3 | 2.74E+09 | 12% | 6 |
| RALA OptiMEM n = 1 | 2.53E+09 | 17% | 6 |
| RALA OptiMEM n = 2 | 1.03E+09 | 15% | 6 |
| RALA OptiMEM n = 3 | 8.63E+08 | 10% | 6 |

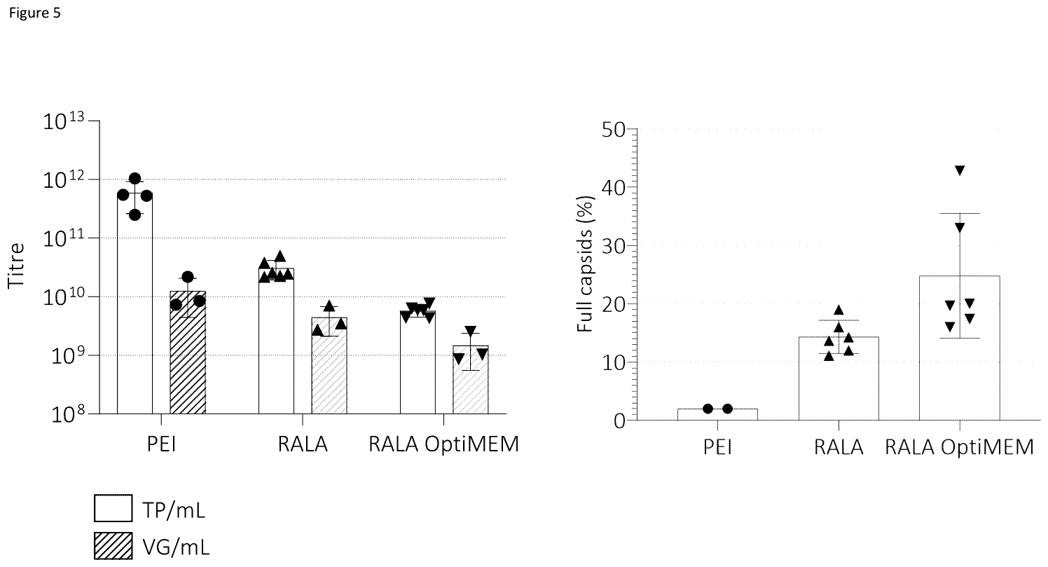

As described in Table 5 and FIG. 5, total particles per mL (as measured using the ELISA assay) reflected qPCR titres with RALA conditions showing lower titres compared with the optimised PEI conditions.

However, the percentage of full capsids was much higher for the RALA transfections than the PEI transfections.

While transfection in RALA OptiMEM reduced the titres compared to the RALA conditions (likely due to a reduced contact time of the particles with the cells), the full capsid percentage was increased in the RALA OptiMEM conditions, possibly due to minimising disruption of the RALA NPs from serum present in the RALA conditions.

TABLE 5
AAV2 titres - genome (VG/mL) and capsid (TP/mL)

| | Run 1 | | | Run 2 | | | Run 3 | | |
|---|---|---|---|---|---|---|---|---|---|
| | | | Full | | | Full | | | Full |
| | VG/mL | TP/mL | capsids (%) | VG/mL | TP/mL | capsids (%) | VG/mL | TP/mL | capsids (%) |
| PEI | 2.19e10 | N/A | N/A | 2.19e10 | 5.28e11 | 1.6* | 2.19e10 | 1.04e12 | 2 |
| | 8.43e9 | N/A | N/A | 8.43e9 | 2.49e11 | 2.95* | 8.43e9 | 5.49e11 | 2 |
| | 7.35e9 | N/A | N/A | 7.35e9 | N/A | N/A | 7.35e9 | N/A | N/A |
| RALA | 3.53e9 | 2.19e10 | 16 | 3.53e9 | 4.89e10 | 14.26 | | | |
| | 7.1e9 | 3.83e10 | 19 | 7.1e9 | 2.58e10 | 13.67 | | | |
| | 2.74e9 | 2.28e10 | 12 | 2.74e9 | 2.47e10 | 11.1 | | | |
| RALA | 2.53e9 | 7.67e9 | 33 | 2.53e9 | 5.91e9 | 48.83 | | | |
| OptiMEM | 1.03e9 | 6.37e9 | 16 | 1.03e9 | 5.91e9 | 17.39 | | | |
| | 8.63e8 | 4.34e9 | 20 | 8.63e8 | 4.39e9 | 19.67 | | | |

N/A - sample too concentrated for total particles readout
*omitted as samples off standard curve

The lower transfection efficiency observed for the RALA conditions may likely arise from the lack of optimisation for these conditions compared with the PEI conditions. The increased % of full viral particles using RALA for transfection compared with PEI indicates that the RALA peptides (under optimised conditions) are likely to increase transfection per producer cell.

### Example 3—Transfection Using RALA Peptides Increases AAV8 Genome and Capsid Production, and the Percentage of Full AAV8 Particles Produced in Adherent HEK293 End-Point Culture Compared with PEI Transfection

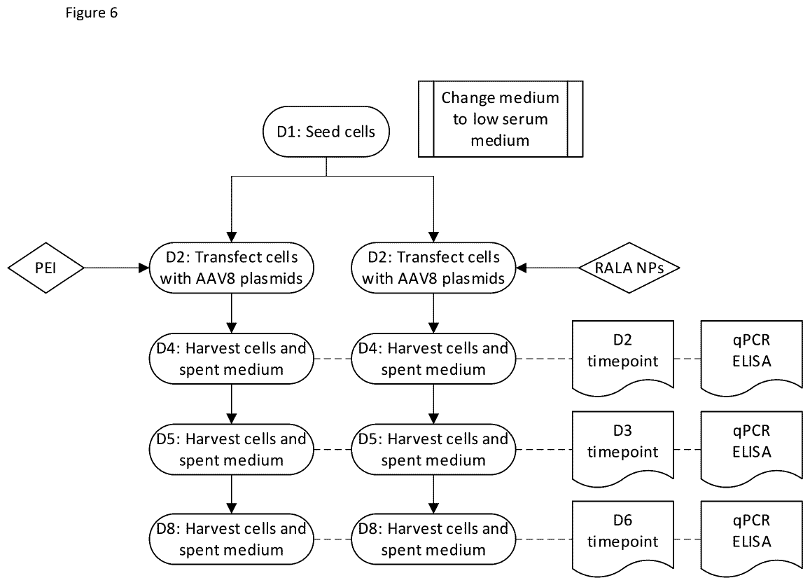

A flow chart of the protocol used is shown in FIG. 6. HEK293T/17 cells were seeded into 6-well plates (1 well/transfection) at a concentration of 1×10⁶ cells/well on day 1 and changed to low serum DMEM medium with 5% FBS. On day 2 cells were transfected for 5 hrs with either RALA nanoparticles (NP:10) or using PEI-Pro (2:1) using 1.56 μg total plasmid per 1×10⁶ cells. The ratio of GFP:RepCap:Helper was kept constant for the different conditions. Samples were terminated via media and lysate collection at 48 hrs, 72 hrs and 120 hrs post transfection.

Viral genomes and viral capsids were quantified using the assays described in Example 1, adapted for AAV8 genome/capsid detection.

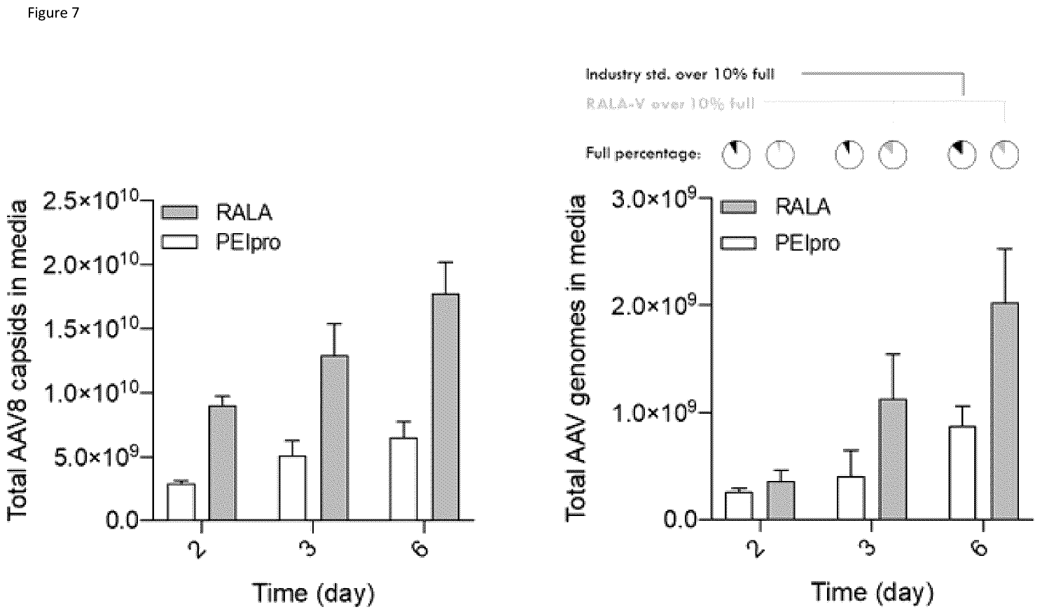

As described in Table 6 and FIG. 7, this experiment showed a significant increase in AAV8 production by RALA particles compared to PEI-Pro, and also showed an increase in full/empty capsid ratio with RALA showing 57% more full AAV particles produced by day 6 than PEI-Pro.

TABLE 6
AAV8 titres - genome (VG/mL) and capsid (TP/mL)

| | | | | Day 3 | | | | | |
|---|---|---|---|---|---|---|---|---|---|
| | | | | | | Full | | | |
| | Day 2 | | | | | capsids | Day 5 | | |
| | VG/mL | TP/mL | Empty:full | VG/mL | TP/mL | (%) | VG/mL | TP/mL | Empty:full |
| PEI | 2.59e8 ± | 2.95e9 ± | 10:1 | 4.05e8 ± | 5.08e9 ± | 12:1 | 8.76e8 ± | 6.51e9 ± | 6:1 |
| | 1.75e7 | 1.93e8 | | 1.41e8 | 1.16e8 | | 1.06e8 | 1.22e9 | |
| RALA | 3.31e8 ± | 9e9 ± | 24:1 | 1.13e9 ± | 1.3e10 ± | 6:1 | 2.02e9 ± | 1.78e10 ± | 8:1 |
| | 5.73e7 | 6.98e8 | | 2.93e8 | 2.41e9 | | 2.93e8 | 2.42e9 | |

Therefore, using optimised conditions, transfection using RALA peptides significantly increased production of AAV8 genomes and capsids, as well as decreasing the ratio of empty:full AAV8 capsids compared with PEI transfection.

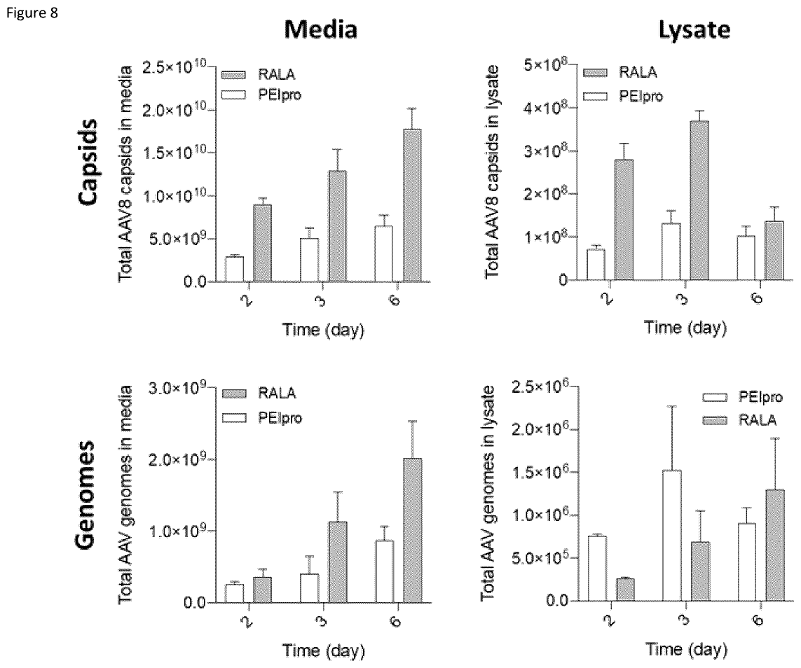

As illustrated in FIG. 8, the bulk of the AAV8 production (both capsids and genomes) was found to be released into the media, with total numbers in cell lysate remaining several orders below the media numbers. This suggests that RALA transfection may lend itself to the continuous production of viral vectors.

### Example 4—Transfection Using RALA Peptides Increases AAV8 Genome and Capsid Production, and the Percentage of Full AAV8 Particles Produced in Adherent HEK293 Continuous Culture Compared with PEI Transfection

Following the results of Example 3, which demonstrated that the bulk of the AAV8 production (both capsids and genomes) was found to be released into the media, a further experimental protocol was devised to determine whether the continuous harvest of AAV8 using RALA transfection was feasible.

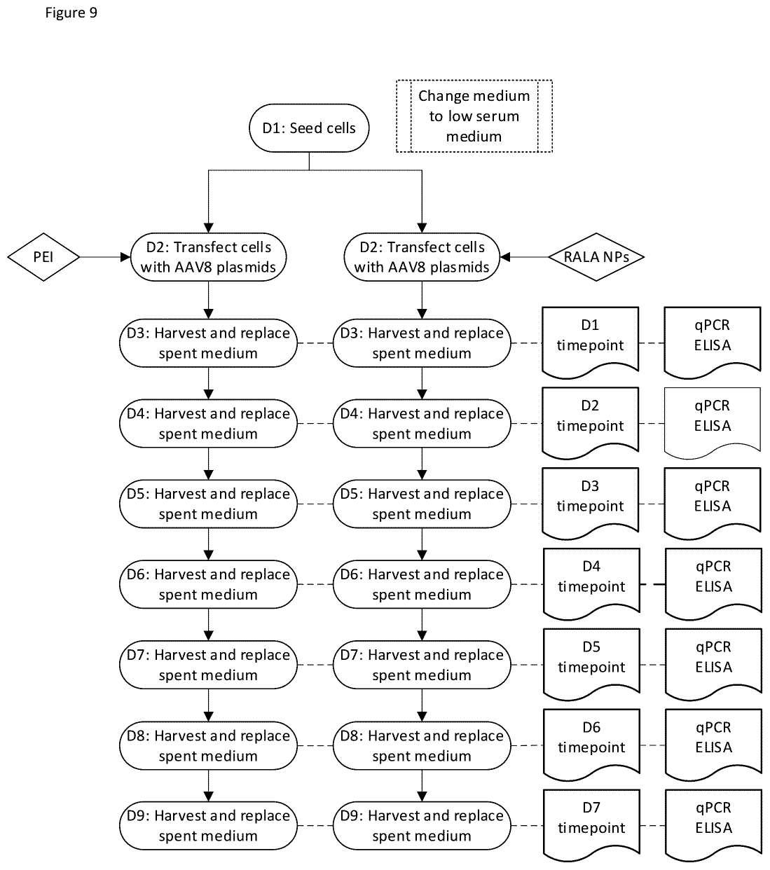

A flow chart of the protocol used is shown in FIG. 9. HEK293T/17 cells were seeded into 6-well plates (1 well/transfection) at a concentration of 1×10⁶ cells/well on day 1 and changed to low serum DMEM medium with 5% FBS. On day 2 cells were transfected for 5 hrs with either RALA nanoparticles (NP:10) or using PEI-Pro (2:1) using 1.56 μg total plasmid per 1×10′ cells. The ratio of GFP:RepCap:Helper ratio was kept constant for the different conditions. Continuous harvesting from the HEK293/17 cells was carried out via collection of media every 24 hrs over a 7-day period.

Viral genomes and viral capsids were quantified using the assays described in Example 1, adapted for AAV8 genome/capsid detection.

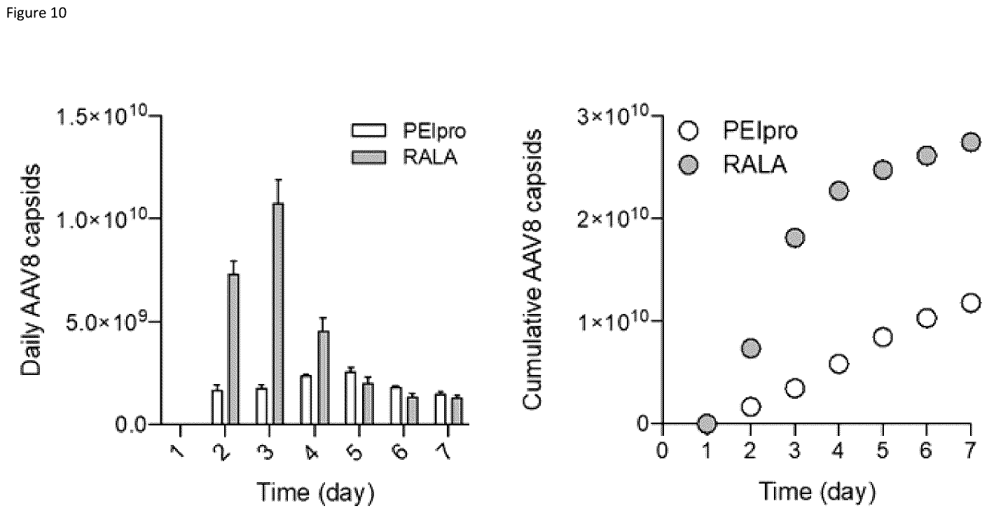

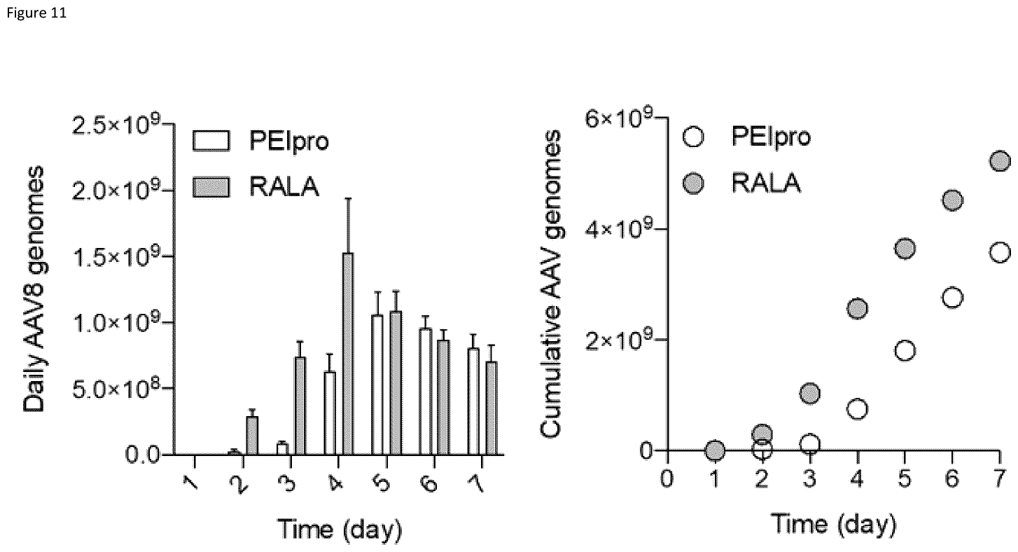

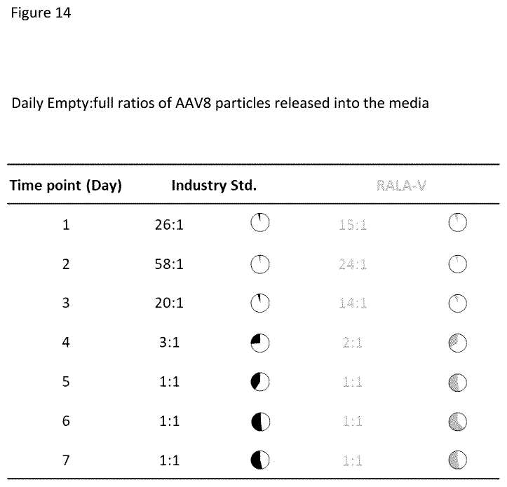

As described in FIG. 10, FIG. 11, FIG. 14, and Table 7, this experiment showed an increase in AAV8 production by RALA particles compared to PEI-Pro (referred to in these tables and Figures as “industry standard”), in particular on days 2, 3 and 4 post transfection. Cumulative harvesting showed a marked increase in production of viral genomes and total capsids by RALA particles compared to PEI-Pro. Alongside increased proportions of full/empty capsids, RALA shows promising results for continuous production of AAV particles.

Based on these studies, it has been shown that transfection using RALA peptides increased AAV particle production under optimised conditions, and even under non-optimised conditions can produce higher full:empty ratios of capsids compared with PEI-Pro transfection.

Further experiments were then designed to assess the utility of RALA peptides for transfection in suspension cultures, and using other types of viral vectors, including other AAV serotypes and lentiviral vectors.

### Example 5—the Decreased Cytotoxicity Observed when Transfecting Using RALA Peptides in Adherent Culture is Maintained for Transfection in Suspension Culture for all AAV and LV Vectors Tested

Having previously observed decreased cytotoxicity when transfecting producer cells with RALA rather than PEI, it was investigated whether this decreased cytotoxicity would be retained when transfection is carried out in suspension culture.

HEK293-F cells were seeded into shake flasks with a 20 mL serum-free BalanCD medium at working volume at a cell concentration of 1×10⁶/mL for lentiviral production and 2×10⁶/mL for AAV production. Transfection with the plasmids for AAV1, AAV2, LV (two experimental replicates, LV1 and LV2) were carried out using PEI-Pro or RALA peptides as set out in Table 8 below.

The HEK293-F cell counts were determined using flow cytometry immediately prior to transfection, and then at 24 hrs, 48 hrs and 72 hrs post transfection. The results are shown in Table 9 below.

TABLE 7
AAV8 titres - genome (VG/mL) and capsid (TP/mL)

| | Day 1 | | | Day 2 | | | Day 3 | | | | |
|---|---|---|---|---|---|---|---|---|---|---|---|
| | | | Full | | | Full | | | Full | | |
| | | | capsids | | | capsids | | | capsids | Day 4 | |
| | VG/mL | TP/mL | (%) | VG/mL | TP/mL | (%) | VG/mL | TP/mL | (%) | VG/mL | TP/mL |
| PEI | 1.33e6 ± | 3.53e7 ± | 26:1 | 2.83e7 ± | 1.68e9 ± | 58:1 | 8.62e7 ± | 1.80e9 ± | 20:1 | 6.35e8 ± | 2.39e9 ± |
| | 1.56e5 | 8.93e6 | | 1.05e7 | 2.37e8 | | 1.96e7 | 1.31e8 | | 1.31e8 | 7.14e7 |
| RALA | 6.61e5 ± | 1.03e7 ± | 15:1 | 2.92e8 ± | 7.36e9 ± | 24:1 | 7.39e8 ± | 1.08 × | 14:1 | 1.53e9 ± | 4.57e9 ± |
| | 9.98e4 | 2.92e6 | | 5.01e7 | 5.81e8 | | 1.16e8 | e10 ± | | 4.05e8 | 5.92e8 |
| | | | | | | | | 1.11e8e6 | | | |

| | Day 4 | Day 5 | | | Day 6 | | | Day 7 | | |
|---|---|---|---|---|---|---|---|---|---|---|
| | Full | | | Full | | | Full | | | Full |
| | capsids | | | capsids | | | capsids | | | capsids |
| | (%) | VG/mL | TP/mL | (%) | VG/mL | TP/mL | (%) | VG/mL | TP/mL | (%) |
| PEI | 3:1 | 1.06e9 ± | 2.60e9 ± | 1:1 | 9.56e8 ± | 1.83e9 ± | 1:1 | 8.12e8 ± | 1.51e9 ± | 1:1 |
| | | 1.76e8 | 1.76e8 | | 8.81e7 | 1.83e7 | | 1.01e8 | 1.00e8 | |
| RALA | 2:1 | 1.09e9 ± | 2.05e9 ± | 1:1 | 8.68e8 ± | 1.38e9 ± | 1:1 | 7.06e8 ± | 1.31e9 ± | 1:1 |
| | | 1.55e8 | 3.01e8 | | 7.53e7 | 1.53e8 | | 1.22e8 | 1.11e8 | |

TABLE 8
Details of transfection/NP preparation

| | | | DNA for 2 | | |
|---|---|---|---|---|---|
| Vector | Cell line | Plasmids | vessels | NP Ratio | n |
| LV1 and LV2 | F line | Gag-Pol | 300 μg | N/A PEI pro ctl | 2 |
| | | LV-GFP | | | |
| | | VSVg | | | |
| | F line | Gag-Pol | 300 μg | NP1 | 2 |
| | | LV-GFP | | | |
| | | VSVg | | | |
| AAV1 | F line | pDG1 | 360 μg | N/A PEI pro ctl | 2 |
| | | pSSGFP | | | |
| | F line | pDG1 | 360 μg | NP1 | 2 |
| | | pSSGFP | | | |
| AAV2 | F line | pDG1 | 360 μg | N/A PEI pro ctl | 2 |
| | | pSSGFP | | | |
| | F line | pDG1 | 360 μg | NP1 | 2 |
| | | pSSGFP | | | |

TABLE 9
HEK293-F cell count and viability results for PEI-Pro and RALA transfection
of HEK293-F cells in suspension with AAV1, AAV2, LV1 and LV2

| | 24 h post-transf | | 48 h post-transf | | 72 h post-transf | |
|---|---|---|---|---|---|---|
| Sample | Via % | Count | Via % | Count | Via % | Count |
| PEI LV 1 | 61.2 | 1.27E+06 | 66.5 | 1.72E+06 | 63.7 | 1.56E+06 |
| PEI LV 2 | 60.3 | 1.34E+06 | 57.1 | 1.42E+06 | 58.6 | 1.53E+06 |
| RALA LV 1 | 92 | 2.31E+06 | 92 | 3.42E+06 | 89.6 | 4.15E+06 |
| RALA LV 2 | 92.7 | 2.05E+06 | 88.6 | 2.83E+06 | 86 | 3.27E+06 |
| PEI AAV1 1 | 90.9 | 3.99E+06 | 80.6 | 3.70E+06 | 61.4 | 3.74E+06 |
| PEI AAV1 2 | 90 | 3.95E+06 | 73.8 | 4.54E+06 | 55.4 | 3.34E+06 |
| RALA AAV1 1 | 90 | 3.63E+06 | 86.1 | 4.14E+06 | 83.1 | 5.82E+06 |
| RALA AAV1 2 | 88.2 | 3.52E+06 | 83.9 | 3.39E+06 | 82.5 | 5.55E+06 |
| PEI AAV2 1 | 90.9 | 3.97E+06 | 85.6 | 4.08E+06 | 73.1 | 4.33E+06 |
| PEI AAV2 2 | 91.2 | 3.95E+06 | 85.8 | 4.16E+06 | 74.4 | 4.83E+06 |
| RALA AAV2 1 * | 87.7 | 3.57E+06 | 81.6 | 3.81E+06 | 76 | 2.52E+06 |
| RALA AAV2 2 | 89.1 | 3.37E+06 | 84.8 | 4.07E+06 | 76.5 | 5.51E+06 |

Average cell density at transfection

| | |
|---|---|
| LV | 2.14E+06 |
| AAV | 3.66E+06 |

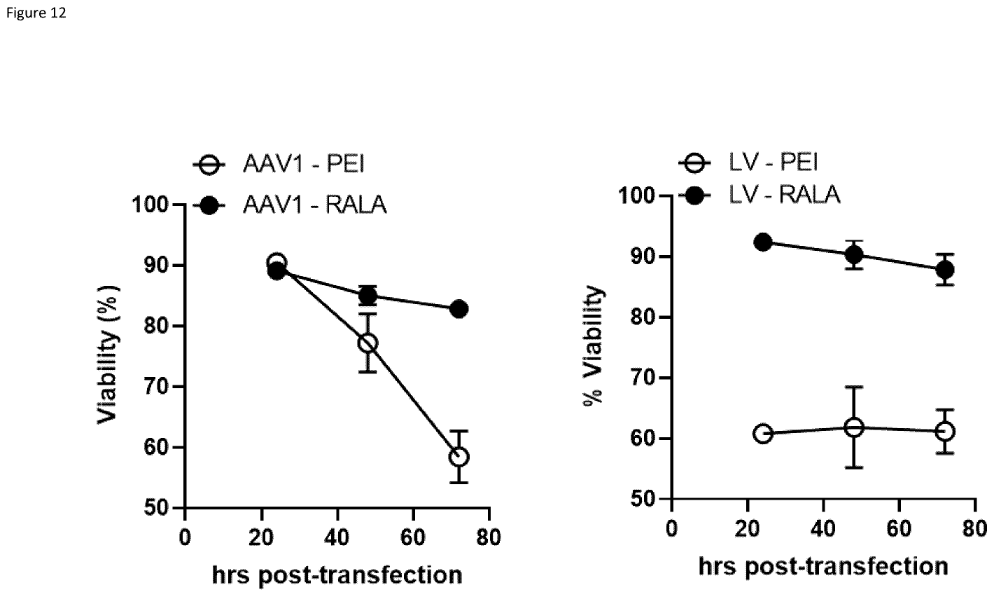

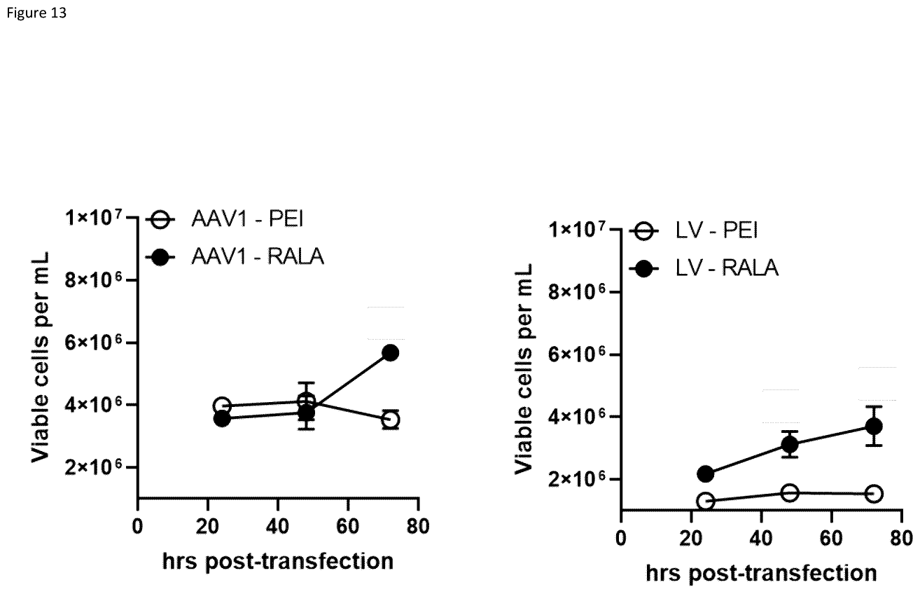

As demonstrated by the results in Table 9 and as illustrated in FIGS. 12 and 13, transfection of HEK293-F cells with the LV2 and LV2 plasmids using PEI resulted in a marked decrease in HEK293-F cell number and viability compared transfection of HEK293-F cells with the LV1 plasmids using RALA peptides. Even after 72 hrs the number of HEK293-F cells had not been restored to pre-transfection levels when PEI was used, whereas with RALA transfection the number of HEK293-F cells had increased compared with pre-transfection levels after 72 hrs, with high levels (>85% viability being maintained).

Similar results were also observed for the AAV1 transfections, with the improvement in HEK293-F cell viability and cell number being even more pronounced compared with LV.

For AAV2 transfections these trends were also observed, although the data for the AAV2 1 data set was outside the acceptance range (*) and as such the AAV2 data were excluded from FIGS. 12 and 13.

Therefore, the results of the cytotoxicity testing for suspension culture were consistent with those previously observed for RALA transfection of adherent producer cells rather than PEI. These data suggest that the advantages associated with RALA transfection (improvements in viral capsid and viral genome production, and particularly the increase in full:empty ratio) as demonstrated for adherent cell culture are maintained in suspension cell culture. Accordingly, further experiments were conducted to investigate whether viral vectors can be successfully produced in suspension culture using RALA peptides.

### Example 6—RALA Peptides Successfully Transfect HEK293-F Cells in Suspension Culture to Produce Lentiviral Vector

HEK293-F cells were grown in suspension culture and transfected with plasmids for the production of LV, AAV1 and AAV2 using RALA peptides as described in Example 5. Samples were terminated via media and lysate collection at 72 hr post transfection.

Viral genomes were quantified using the assays described in Example 1, adapted for LV, AAV1 or AAV2 genome detection as appropriate.

As described in Table 10, this experiment showed that LV, AAV1 and AAV2 viral genomes were produced following RALA transfection. Therefore, RALA peptides can be used successfully to transfect HEK293-F cells in suspension culture to produce LV, AAV1 and AAV2 vectors.

TABLE 10
LV, AAV1 and AAV2 genome titres (VG/mL)
by RT-ddPCR and qPCR respectively

| Sample | Vg/mL | % CV | Replicates |
|---|---|---|---|
| LV1 -RALA | 8.87E+07 | 13.80 | 12 |
| LV2 -RALA | 6.73E+07 | 9.39 | 9 |
| AAV1-RALA 1 | 2.19E+08 | 12 | 3 |
| AAV1-RALA 2 | 2.39E+08 | 4 | 3 |
| AAV2-RALA 1 | 5.82E+08 | 6 | 3 |
| AAV2-RALA 2 | 5.19E+08 | 10 | 3 |

Given the advantages of RALA-based transfection demonstrated in adherent cell culture, and the fact at least some of these benefits (namely decreased cytotoxicity) have been shown to be maintained (Example 4) when switching to suspension culture, these data suggest that RALA-based transfection has the potential to allow for the production of viral vectors at high titres and of high quality (high full:empty ratio). Combined with the fact that successful continuous production of viral vectors has also been demonstrated (Example 3), the experiments reported herein demonstrated the potential for continuous suspension-based production of viral vectors using RALA peptides. Such a method, particularly one which produces viral vectors at high titres and with increased full:empty vector ratios compared with conventional protocols offers significant advantages for the commercial production of viral vectors.

1. A method of producing a viral vector comprising, transfecting a producer cell with one or more nucleic acids using an amphipathic cell penetrating peptide, wherein the peptide is:
a) less than approximately 50 amino acid residues in length; and
b) has at least 6 arginine residues (R), at least 12 alanine residues (A), at least 6 leucine residues (L), optionally at least one cysteine residue (C), and at least two but no greater than three glutamic acids (E);
wherein:
(i) the arginine (R) residues are evenly distributed along the length of the peptide;
the ratio of arginine (R) to negatively charged glutamic acid (E) residues is from at least 6:2 to 9:2 or 6:2 to 8:2;
the ratio of hydrophilic amino acid residues to hydrophobic amino acid residues at pH 7 is at least 30:67 to 40:60 or 30:70 to 40:60; and
said peptide comprises one of the following amino acid sequences:
(SEQ ID No: 1)
WEARLARALARALARHLARALARALRACEA
(SEQ ID No: 2)
WEARLARALARALARLARALARALRACEA
(SEQ ID No: 3)
WEARLARALARALARLARALARALRACEA
(SEQ ID No: 4)
WEARLARALARALARELARALARALRACEA
(SEQ ID No: 5)
REARLARALARALARLARALARALRACEA
(SEQ ID No: 6)
REARLARALARALARLARALARALRAREA
(SEQ ID NO: 7)
REARLARALARALARELARALARALRAREA
or a fragment thereof;
(ii) said peptide comprises WEARLARALARALARELARALARALRACEA (SEQ ID No. 4); or
(iii) said peptide comprises a peptide with at least 80% sequence identity to SEQ ID NO: 1, or a fragment thereof.

2. The use method of claim 1, wherein the peptide comprises SEQ ID NO: 1, 2, 5, or a fragment thereof.

3. (canceled)

4. The method of claim 1, comprising
harvesting the viral vectors produced by the transfected producer cells.

5. The method of claim 4, wherein the peptide is complexed with the one or more nucleic acid to form a nanoparticle prior to transfection.

6. The method of claim 5, wherein:
a) each one or more nucleic acid is complexed separately with the peptide to form nanoparticles; or
b) the one or more nucleic acids are pooled prior to complexing with the peptide to form nanoparticles.

7. The method of claim 1, wherein the one or more nucleic acids are comprised in one or more plasmids.

8. The method of claim 1, wherein:
a) the transfection efficiency is at least about 40%; and/or
b) the transfection is at least two times more efficient compared with a corresponding transfection using polyethylenimine (PEI).

9. The method of claim 1, wherein:
a) the peptide results in cytotoxicity of less than 20% to the producer cells; and/or
b) the peptide is at least 10 times, less toxic to the producer cells than PEI.

10. The method of claim 1, wherein the number of viral capsids produced is at least 1.5-fold greater than the number of viral capsids produced compared with a corresponding transfection using PEI.

11. The method of claim 1, wherein:
a) the ratio of empty:full viral particles is about less than about 20:1; and/or
b) the transfection results in the production of at least about 30% more full viral particles compared with a corresponding transfection using PEI.

12. The method of claim 1, wherein the viral vector is an adeno-associated virus (AAV) vector, a lentiviral vector, a retroviral vector, an adenoviral vector, a herpes-simplex viral vector, a poxvirus vector, or a baculovirus vector.

13. (canceled)

14. The method of claim 12, wherein the viral vector is an AAV vector, and the AAV vector is an AAV2, AAV8, AAV1, AAV3, AAV4, AAV5, AAV6, AAV7, AAV8, AAV9, AAV10 or AAV11 serotype.

15. (canceled)

16. The method of claim 7, wherein the one or more plasmid comprises (i) a plasmid comprising the RepCap genes; (ii) a plasmid comprising a transgene of interest; and (iii) a helper plasmid.

17. The method of claim 1, wherein the transfecting of the producer cells is carried out in serum-free or low-serum medium, and/or the population of producer cells is cultured and transfected in a suspension culture system or an adherent culture system.

18. (canceled)

19. The method of claim 1, wherein the producing of the viral vector is a continuous production.

20. The method of claim 1 further comprising one or more of the following steps:
a) expanding the population of producer cells prior to transfection;
b) culturing the population of producer cells prior to transfection;
c) changing the medium of the producer cells prior to transfection;
d) changing the medium of the producer cells after transfection; and/or
e) purification of the viral vectors.

21. (canceled)

22. The method of claim 1, wherein the method does not comprise a step of polishing the viral vectors.

23. A population of viral vectors obtained by the method of claim 4.

24. The population of viral vectors of claim 23, wherein the ratio of empty:full viral particles is about less than about 20:1.

25. A composition comprising one or more nucleic acids and an amphipathic cell penetrating peptide, wherein the amphipathic cell penetrating peptide is:
a) less than approximately 50 amino acid residues in length; and
b) has at least 6 arginine residues (R), at least 12 alanine residues (A), at least 6 leucine residues (L), optionally at least one cysteine residue (C), and at least two but no greater than three glutamic acids (E);
wherein:
(i) the arginine (R) residues are evenly distributed along the length of the peptide;
the ratio of arginine (R) to negatively charged glutamic acid (E) residues is from at least 6:2 to 9:2 or 6:2 to 8:2;
the ratio of hydrophilic amino acid residues to hydrophobic amino acid residues at pH 7 is at least 30:67 to 40:60 or 30:70 to 40:60; and
said peptide comprises one of the following amino acid sequences:
(SEQ ID No: 1)
WEARLARALARALARHLARALARALRACEA
(SEQ ID No: 2)
WEARLARALARALARLARALARALRACEA
(SEQ ID No: 3)
WEARLARALARALARLARALARALRACEA
(SEQ ID No: 4)
WEARLARALARALARELARALARALRACEA
(SEQ ID No: 5)
REARLARALARALARLARALARALRACEA
(SEQ ID No: 6)
REARLARALARALARLARALARALRAREA
(SEQ ID NO: 7)
REARLARALARALARELARALARALRAREA
or a fragment thereof;
(ii) said peptide comprises WEARLARALARALARELARALARALRACEA (SEQ ID No. 4); or
(iii) said peptide comprises a peptide with at least 80% sequence identity to SEQ ID NO: 1, or a fragment thereof.


SEQUENCE LISTING (7 sequences)

SEQ ID NO: 1 - protein, 30 aa, Artificial Sequence
  RALA peptide
WEARLARALARALARHLARALARALRACEA

SEQ ID NO: 2 - protein, 29 aa, Artificial Sequence
  RALA peptide
WEARLARALARALARLARALARALRACEA

SEQ ID NO: 3 - protein, 29 aa, Artificial Sequence
  RALA peptide
WEARLARALARALARLARALARALRACEA

SEQ ID NO: 4 - protein, 30 aa, Artificial Sequence
  RALA peptide
WEARLARALARALARELARALARALRACEA

SEQ ID NO: 5 - protein, 29 aa, Artificial Sequence
  RALA peptide
REARLARALARALARLARALARALRACEA

SEQ ID NO: 6 - protein, 29 aa, Artificial Sequence
  RALA peptide
REARLARALARALARLARALARALRAREA

SEQ ID NO: 7 - protein, 30 aa, Artificial Sequence
  RALA peptide
REARLARALARALARELARALARALRAREA